(19) United States
(12) Patent Application Publication
(10) Pub. No.: US 2003/0109682 A1
(43) Pub. Date: Jun. 12, 2003

(54) Maytansines and maytansine conjugates

(76) Inventors: Daniel Santi, San Francisco, CA (US); David C. Myles, Kensington, CA (US); Brian Metcalf, Moraga, CA (US); C. Richard Hutchinson, San Mateo, CA (US); Gary Ashley, Alameda, CA (US)

Correspondence Address: Carolyn A. Favorito, Morrison & Foerster LLP, Suite 500, 3811 Valley Centre Drive, San Diego, CA, 92130-2332, US

(21) Appl. No.: 10/235,228

(22) Filed: Sep. 3, 2002

Related U.S. Application Data
(60) Provisional application No. 60/318,206, filed on Sep. 7, 2001.

(51) Int. Cl. C07K 16/46

(52) U.S. Cl. 530/391.1; 540/462

(57) ABSTRACT

Cytotoxin-targeting molecule conjugates comprising cytotoxin and an antibody, growth factor, or polysaccharide together with a pH-sensitive or redox potential-sensitive linker. Novel ansamitocins and recombinant genes and organisms that produce them. The use of the described conjugates in the treatment of cancer and other hyperproliferation diseases.

### CROSS-REFERENCE TO RELATED APPLICATIONS

This application claims priority to U.S. patent application Serial No. 60/318,206, filed Sep. 7, 2001, which is incorporated herein by reference.

### BACKGROUND

Maytansine (1) is an ansa-macrolide derived primarily via the polyketide biosynthetic pathway. Maytansine was isolated by Kupchan and coworkers in 1972 from the African plant, Maytenus ovatus (Celastraceae), later renamed Maytenus serrata, on the basis of its cytotoxicity against KB cells and its antileukemic activity against the mouse P388 lymphocytic leukemia. Maytansine shows high cytotoxic activity against cultured KB cells, with ED₅₀=8.8 pM. Maytansine is an antimitotic agent acting as an inhibitor of tubulin polymerization, thus interfering with the formation of microtubules in the cell nucleus. Maytansine also inhibits DNA, RNA, and protein synthesis, with the greatest effect being seen on DNA synthesis.

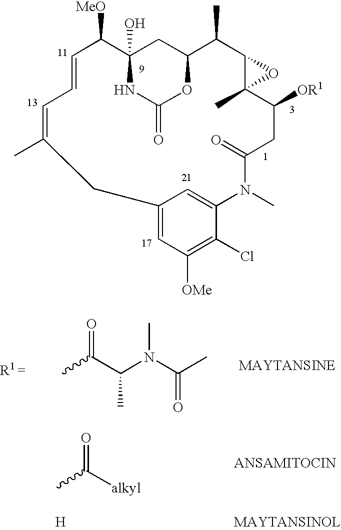

The low yield of maytansine from the original source (0.2 mg/kg dried plant material) led to a search for a better producing organism. Of these new organisms, Maytenus buchananii was found to provide maytansine in a yield of 1.5 mg/kg of dried plant material, but maytansine proved extremely expensive even at this level of production. It was subsequently discovered that species of the actinomycete Actinosynnema produce compounds related to maytansine called ansamitocins, in which the N-acetyl-N-methyl-(L)-alanine ester at C3 is replaced by simple fatty acid esters. While the ansamitocins are not as potent as maytansine in in vitro cytotoxicity assays, they are still cytotoxic and also serve as starting materials for the synthesis of maytansinol, the C3-alcohol. Maytansinol is converted into maytansine by chemical acylation.

Phase I clinical trials with maytansine showed encouraging responses in patients with acute lymphocytic leukemia, breast carcinoma, ovarian cancer, thymoma, melanoma, and non-small scale lung cancer. Phase II trials revealed that the dose-limiting toxicity of maytansine was such as to preclude effective clinical use. Maytansine showed manageable gastrointentinal toxicity, but neurotoxicity at the site of administration resulted in pain of such magnitude as to prevent further administration.

The use of cytotoxins in treating cancer is thus complicated by non-specific toxicities. One approach to improving the target selectivity of cytotoxins is to conjugate them to molecules that target the cytotoxins to desired cell types. Typically, the targeting molecules are antibodies directed against a cell-surface protein that is characteristic of the desired cell type. Other targeting molecules, including serum proteins, polysaccharides, and synthetic polymers have also been used. Once bound to the target cell antigen, the conjugate is taken into the cell through endocytosis. The cytotoxins used include DNA alkylating agents such as nitrogen mustards, doxorubicin, 5-fluorouridine, vinblastin, nucleoside antimetabolites, and protein toxins such as ricin, abrin, saporin, gelonin, Pseudomonas exotoxin, and diphtheria toxin. General protocols for the design and use of conjugated antibodies are described in Monoclonal Antibody-Based Therapy of Cancer by Michael L. Grossbard, ed. (1998) (incorporated herein by reference).

Once delivered to the target cell by the targeting molecule, the cytotoxin typically must be released from the conjugate in order to function. This release is often the result of a cleavable linkage between the targeting molecule and the cytotoxin. The linkage is stable under extracellular conditions, such that the cytotoxin-targeting molecule conjugate can be stored and safely administered, but is unstable upon reaching the target cell. This requires a linkage that is sensitive towards conditions specific to the environment inside the target cell.

Endocytosis into the cell is a complex process. Invaginations on the cell surface close to form endosomes, that are taken into the cell. Drug conjugates can be trapped inside endosomes either while in solution (fluid-phase endocytosis), after non-specific binding to the cell surface (absorptive endocytosis), or after specific binding to a cell-surface receptor that subsequently becomes enclosed in the endosome (receptor-mediated endocytosis). Any or all of these mechanisms can be used to bring drug conjugates into the cell. Depending upon the process involved, the endosomes either fuse with particular cell organelles such as the Golgi apparatus, recycle to the cell surface, or form primary or secondary lysosomes.

The environment inside the endosomes and lysosomes is characteristically acidic, and contains a number of specific digestive enzymes. The release of the cytotoxin from the conjugate inside lysosomes may thus take advantage of either specific enzymatic cleavage or the pH difference between the extracellular and lysosomal environments. While the pH of blood is typically about 7.3 to 7.4, the pH in the endosome is 5.0 to 6.5, and the pH in the lysosome is about 4.0, and can be as low as 3.8 at early stages of digestion. The intracellular environment inside tumor tissue has been measured to be 0.5 to 1.0 pH units lower than in normal tissue as well. These pH differentials form the basis for therapy using cytotoxin-antibody conjugates in which the cytotoxin and the antibody are connected using a pH-sensitive linker. The antibody targets the cytotoxin to a particular cell or tissue, where upon endocytosis into the acidic environment of the lysosome, the conjugate is cleaved to release the cytotoxin.

The use of pH-sensitive linkages based on aconitic acid derivatives has been described in Shen and Ryser, “Acidity-sensitive spacer molecule to control the release of pharmaceuticals from molecular carriers,” U.S. Pat. Nos. 4,631,190 and 5,144,011, and “Cis-aconityl spacer between daunomycin and macromolecular carriers: a model of pH-sensitive linkage releasing drug from a lysomotropic conjugate,” Biochem. Biophys. Res. Comm. (1981) 102:1048-1054 (each of which is incorporated herein by reference). Other examples of pH-sensitive linkers are described in Kratz et al., “Drug-polymer conjugates containing acid-cleavable bonds,” Critical Reviews in Therapeutic Drug Carrier Systems (1999) 16: 245-288 (incorporated herein by reference).

Despite its promise as an anticancer agent, the high non-selective toxicity of maytansine led to its being dropped from clinical trials. There thus exists a need for maytansine analogs that can be administered in an inactive pro-drug form but which can be released in active form upon reaching their target, so as to minimize the neurotoxicity at the site of administration as well as other non-specific toxicities associated with such a powerful antimitotic agent.

### SUMMARY OF THE INVENTION

The present invention provides novel compounds useful in the treatment of diseases of hyperproliferation. In one aspect of the invention, novel pH-sensitive and redox-sensitive linkers are provided that allow conjugation of a cytotoxin to a molecule that directs the cytotoxin to a particular cell, tissue, or organ, herein known as a “targeting molecule.” In a second aspect of the invention, novel analogs of maytansine are provided as well as recombinant genes and organisms that produce them. In a third aspect of the invention, novel conjugates between cytotoxins and targeting molecules are provided. In a fourth aspect of the invention, methods to treat diseases of hyperproliferation are provided.

Thus in one aspect of the invention, novel pH-sensitive linkers are provided. These linkers constitute a means of conjugating a cytotoxic agent to a targeting molecule, where the cytotoxic agent is released from the conjugate upon uptake into a cellular compartment of sufficiently low pH. The linker is a vicinal dicarboxylic acid derivative wherein one acid is linked to the amine of an aminoalcohol through an amide bond. The linker is attached to the targeting molecule through a thiol or a third carboxylate functionality. The cytotoxic agent is linked through the alcohol moiety, so as to produce an acetal-type linkage. Upon hydrolysis of the amide, the liberated ammonium functionality acts as an intramolecular acid catalyst to accelerate the hydrolysis of the acetal linkage, thus freeing the cytotoxic agent from the conjugate.

In one embodiment, the linker is a vicinal dicarboxylic acid coupled to a 1,ω-aminoalkanol. In a second embodiment, the linker is a vicinal dicarboxylic acid coupled to a 1-(hydroxymethyl)aryl amine. In a third embodiment, the linker is a vicinal dicarboxylic acid coupled to a 1-(aminomethyl)aryl alcohol, 1-(aminomethyl)aryl amine, or 1-(aminomethyl)aryl thiol.

In other embodiments, novel acyl-acetal linkers are provided. The linker comprises a carbonate, carbamate, urea, thiocarbonate, thiourea, or thiocarbamate functionality attached via an acetal-type linkage to the cytotoxin as well as being attached to the targeting molecule through a moiety which, upon exposure to low pH, liberates a functionality which accelerates the release of the cytotoxin from the conjugate. In one embodiment, the carbonate, carbamate, urea, thiocarbonate, thiourea, or thiocarbamate functionality is attached to the targeting molecule through the alcohol of a 1,ω-aminoalkanol, the amine functionality of which is attached to a group containing a vicinal dicarboxylic acid and a third functionality as described above. Liberation of the ammonium group, as described above, results in accelerated release of the cytotoxin. In a second embodiment, the carbonate, carbamate, urea, thiocarbonate, thiourea, or thiocarbamate functionality is attached to the targeting molecule through the alcohol of a 4-hydroxy-6-methyl-5-heptenoic acid, the carboxylate of which is attached to the targeting molecule via an amide linkage.

In other embodiments of the invention, novel redox potential-sensitive linkers are provided. In one embodiment, the redox potential-sensitive linker is an alkyl-aryl mixed disulfide wherein the aryl moiety is substituted so as to control the steric and electronic properties of the disulfide. Such linkers provide a means of attenuating the rate of thiol-disulfide interchange such that the linkage is stable in environments of low reductive potential, for example in the extracellular environment, but is cleaved in the presence of high concentrations of reducing thiols, for example in the intracellular environment.

In another aspect of the invention, novel maytansine analogs are provided, as well as recombinant maytansine and/or ansamitocin biosynthetic gene clusters and organisms that produce the analogs. In one embodiment, 11-hydroxymaytansine analogs and 13-hydroxymaytansine analogs are provided by inactivation of the dehydratase domain in module 3 or module 2, respectively, of the maytansine and/or ansamitocin polyketide synthase. In another embodiment, maytansine analogs having hydroxyl groups at C17, C18, C19, or C21 are provided by supplying the appropriate starter unit analog to a culture of a mutant of the producing organism deficient in production of the natural starter unit. In other embodiments, the genetically engineered biosynthetic genes, enzymes, and producing organisms are provided.

In another aspect of the invention, novel cytotoxin-targeting molecule conjugates are provided. In one embodiment, the cytotoxin is a novel maytansine or ansamitocin of the invention linked to an antibody, growth factor, or polysaccharide through a novel pH-sensitive or redox-sensitive linker. In a second embodiment, the cytotoxin is a molecule known in the art linked to an antibody, growth factor, or polysaccharide through a novel pH-sensitive or redox-sensitive linker. In a third embodiment, the cytotoxin is a novel maytansine or ansamitocin of the invention linked to an antibody, growth factor, or polysaccharide through a pH-sensitive or redox-sensitive linker known in the art.

In other embodiments of the invention, novel cytotoxin-targeting molecule conjugates are provided wherein multiple molecules of the cytotoxin are attached to each molecule of the targeting molecule through a dendrimer. Each molecule of cytotoxin is linked to a dendrimer via a novel pH-sensitive or redox potential-sensitive linker, and the targeting molecule is either stably connected to the dendrimer or is linked to the dendrimer through novel pH- or redox-sensitive linkers. In one embodiment, the cytotoxin is a novel maytansine or ansamitocin of the invention, linked to the dendrimer using novel linkers. In a second embodiment, the cytotoxin is a molecule known in the art, linked to the dendrimer using novel linkers. In a third embodiment, the cytotoxin is a novel maytansine or ansamitocin of the invention, linked to the dendrimer using linkers known in the art.

### BRIEF DESCRIPTION OF THE DRAWINGS

FIG. 1 illustrates the preparation of an ansamitocin-antibody conjugate, wherein the maytansine is linked through a C9-acetal.

FIG. 2 illustrates the pH-sensitive cleavage of the ansamitocin-antibody conjugate of FIG. 1 at low pH.

FIG. 3 illustrates the preparation of other types of pH-sensitive linkers based on benzylic amino alcohols.

FIG. 4 illustrates the preparation and pH-sensitive cleavage of another type of ansamitocin-antibody conjugate wherein the linkage is through a C9-carbonate.

FIG. 5 shows the preparation of an ansamitocin-antibody conjugate using a redox potential-sensitive linker.

FIG. 6 shows the anticipated organization of modules within the ansamitocin polyketide synthase.

FIG. 7 shows the production of 13-hydroxyansamitocin analogs produced by DH domain inactivations in module 2 of the ansamitocin polyketide synthase.

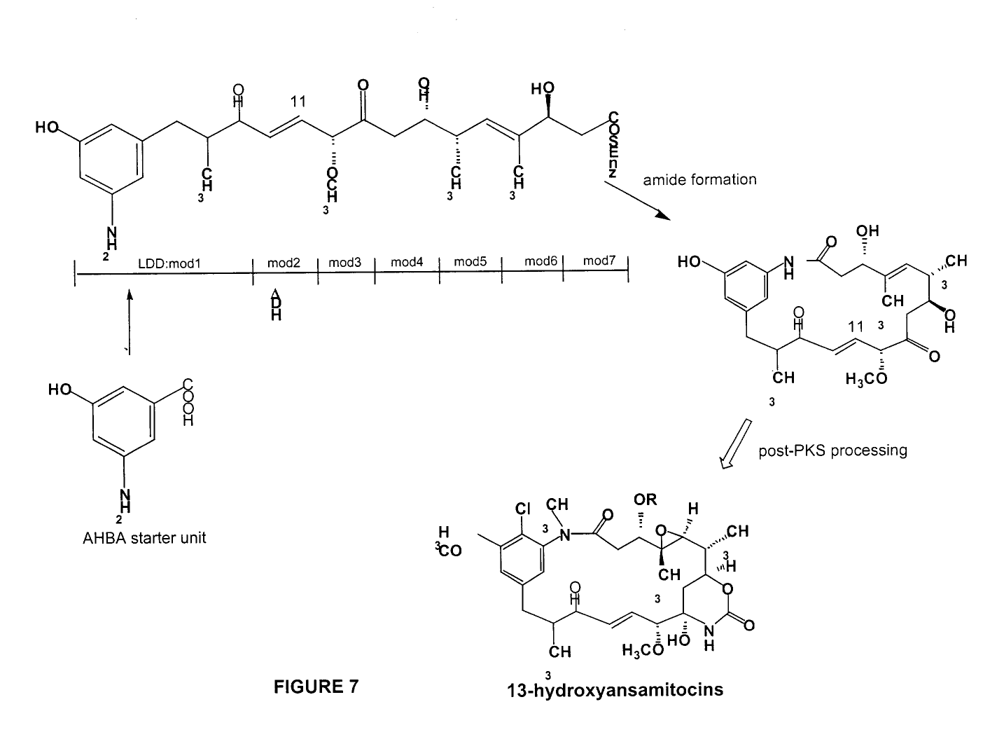

### DETAILED DESCRIPTION OF THE INVENTION

Definitions

The definitions of terms used herein are listed below. The definitions apply to the terms as they are used throughout this specification, unless otherwise limited in specific instances, either individually or as part of a larger group.

As used herein, the terms “maytansines,” “maytansine analogs,” “maytansinoids,” “ansamitocins,” and “ansamitocin analogs” refer to maytansine, ansamitocins, and related compounds of the general formula

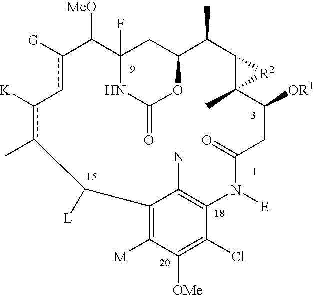

wherein R¹ is H or acyl; R² is O or a bond; E is H or C₁-C₄ alkyl; F is an oxygen, nitrogen, or sulfur atom; G and K are independently H or OH; L is H or OH; and M and N are independently H, OH, or NH₂. Preferred examples of ansamitocins include but are not limited to maytansine, maytanbutine (R⁴=isopropyl), maytanprine (R⁴=ethyl), maytanvaline (R⁴=isobutyl), maytansinol, ansamitocin P0, ansamitocin P1, ansamitocin P2, ansamitocin P3, ansamitocin P3′, and ansamitocin P4.

As used herein, the term “linker” refers to a moiety that connects a first molecule to a second molecule through chemical bonds. In linkers of the invention, the connection can be severed so as to release a biologically active form of the first and/or second molecule. A preferred example of a linker is a moiety that comprises a bond that is stable at neutral pH but is readily cleaved under conditions of low pH. Particularly preferred examples of linkers are moieties that comprise a bond that is stable at pH values between 7 and 8 but is readily cleaved at pH values between 4 and 6. Another example of a linker is a moiety that comprises a bond that is readily cleaved in the presence of an enzyme. Preferred examples of such enzyme-sensitive linkers are peptides comprising a recognition sequence for an endosomal peptidase. Another example of a linker is a redox potential-sensitive linker that is stable under conditions of low reduction potential (e.g., low thiol or glutathione concentration) but cleaved under conditions of high reduction potential (e.g., high thiol or glutathione concentration). Preferred examples of such redox potential-sensitive linkers include disulfides and sulfenamides. Particularly preferred examples include substituted aryl-alkyl disulfides in which the aryl group is substituted with sterically-demanding and electron-withdrawing or electron-donating substitutents, so as to control the sensitivity of the disulfide linkage towards reaction with thiol. Another example of a linker is a moiety that comprises a bond that is readily cleaved upon exposure to radiation. Preferred examples of such radiation-sensitive linkers are 2-nitrobenzyl ethers that are cleaved upon exposure to light. Particularly preferred examples of linkers are moieties that mask the biological activity of one of the two linked molecules until the linkage is severed.

As used herein, the term “dendrimer” refers to a linker comprising multiple points of attachment for molecule. Examples of dendrimers are co-oligomers of diamines and acrylic acids. Preferred examples of dendrimers are Starburst® (PAMAM) molecules with multiple surface amino, hydroxyl, or carboxyl groups, and dendrimers of polylysine. Polylysine dendrimers may be treated with ethylene episulfide to create a dendrimer having multiple surface thiol groups.

As used herein, the term “targeting molecule” refers to a molecule having a specificity for a particular cell, tissue, or organ. Preferred examples of targeting molecules include but are not limited to antibodies, growth factors, and polysaccharides. Particularly preferred examples of targeting molecules include but are not limited to antibodies, growth factors, and polysaccharides that are ligands for cell-surface receptors.

As used herein, the term “aliphatic” refers to saturated and unsaturated straight chain, branched chain, cyclic, or polycyclic hydrocarbons that may be optionally substituted at one or more positions as defined below. Illustrative examples of aliphatic groups include alkyl, alkenyl, alkynyl, cycloalkyl, cycloalkenyl, and cycloalkynyl groups. The term “alkyl” refers to an optionally substituted straight or branched chain saturated hydrocarbon substituent. “Alkenyl” refers to an optionally substituted straight or branched chain hydrocarbon substituent with at least one carbon-carbon double bond. “Alkynyl” refers to an optionally substituted straight or branched chain hydrocarbon substituent with at least one carbon-carbon triple bond.

The term “aryl” refers to optionally substituted monocyclic or polycyclic groups, that optionally include one or more heteroatoms and preferably one to fourteen carbon atoms, and have at least one aromatic ring structure. Aryl groups may be optionally substituted at one or more positions. Illustrative examples of aryl groups include but are not limited to: furanyl, imidazolyl, indanyl, indolyl, indazolyl, isoxazolyl, isoquinolyl, naphthyl, oxazolyl, oxadiazolyl, phenyl, pyrazinyl, pyridyl, pyrimidinyl, pyrrolyl, pyrazolyl, quinolyl, quinoxalyl, tetrahydronaphthyl, tetrazolyl, thiazolyl, thienyl, and the like.

The aliphatic and aryl moieties may be optionally substituted with one or more substituents, preferably from one to five substituents, more preferably from one to three substituents, and most preferably from one to two substituents. The definition of any substituent or variable at a particular location in a molecule is independent of its definitions elsewhere in that molecule unless otherwise indicated. It is understood that substituents and substitution patterns on the compounds of this invention can be selected by one of ordinary skill in the art to provide compounds that are chemically stable and that can be readily synthesized by techniques known in the art as well as those methods set forth herein. Examples of suitable substitutents include but are not limited to: aliphatic, haloaliphatic, halogen, aryl, hydroxy, alkoxy, aryloxy, azido, thio, alkylthio, arylthio, amino, alkylamino, arylamino, acyl, carbamoyl, alkylsulfonyl, sulfonyl, sulfonamido, nitro, cyano, carboxy, guanidine, and the like.

The term “haloaliphatic” refers to an aliphatic group substituted by one or more halogens.

The terms “halo, “halogen,” or “halide” refer to fluorine, chlorine, bromine, and iodine.

The term “acyl” refers to —C(═O)R, where R is an aliphatic group.

The term “alkoxy” refers to —OR, where R is an aliphatic group.

The term “aryloxy” refers to —OR, where R is an aryl group.

The term “carbamoyl” refers to —O(C═O)NRR′, where R and R′ are independently H, aliphatic, or aryl groups.

The term “alkylamino” refers to —NHR, where R is an alkyl group. The term “dialkylamino” refers to —NRR′, where both R and R′ are alkyl groups.

The term “hydroxyalkyl” refers to —R—OH, where R is an aliphatic group.

The term “aminoalkyl” refers to —R—NH₂, where R is an aliphatic group. The term “alkylaminoalkyl” refers to —R—NH—R′, where both R and R′ are aliphatic groups. The term “arylaminoalkyl” refers to R—NH—R′, where R is an is an aryl group.

The term “oxo” refers to a carbonyl oxygen (═O).

The term “isolated” as used herein to refer to a compound of the present invention, means that the compound is in a preparation in which the compound forms a major component of the preparation, such as constituting about 50%, about 60%, about 70%, about 80%, about 90%, about 95%, or more by weight of the components in the preparation.

The term “subject” as used herein, refers to an animal, preferably a mammal, and most preferably a human, that has been the object of treatment, observation, and/or experiment.

The term “therapeutically effective amount” as used herein, means that amount of active compound or pharmaceutical agent that elicits the biological or medicinal response in a cell culture, tissue system, animal, or human that is being sought by a researcher, veterinarian, clinician, or medical doctor, which includes alleviation of the symptoms of the disease or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable salt” refers to a salt of an inventive compound suitable for pharmaceutical formulation. Suitable pharmaceutically acceptable salts include acid addition salts which may, for example, be formed by mixing a solution of a compound with a solution of a pharmaceutically acceptable acid such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. Where a compound carries one or more acidic moieties, pharmaceutically acceptable salts may be formed by treatment of a solution of the compound with a solution of a pharmaceutically acceptable base, such as lithium hydroxide, sodium hydroxide, potassium hydroxide, tetraalkylammonium hydroxide, lithium carbonate, sodium carbonate, potassium carbonate, ammonia, alkylamines, or the like.

The term “pharmaceutically acceptable carrier” refers to a medium that is used to prepare a dosage form of a compound. A pharmaceutically acceptable carrier includes solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), both of which are incorporated herein by reference in their entireties, disclose various carriers used in formulating pharmaceutical compositions and known techniques for the preparation thereof.

The term “pharmaceutically acceptable ester” refers to an ester that hydrolyzes in vivo to produce a compound or a salt thereof. Illustrative examples of suitable ester groups include but are not limited to formates, acetates, propionates, butyrates, succinates, and ethylsuccinates.

The term “inhibitor” refers to a compound that binds to a target protein and in so doing interferes with the natural biological activity of that target. For example, a compound that binds to an enzyme and in so doing blocks the catalytic activity of that enzyme is an inhibitor of that enzyme. The binding may occur at the active site or at a location distal to the active site. A compound that binds to a target that has no known enzymatic activity, for example a structural protein, and in doing so prevents the normal biological function of the target from being realized is an inhibitor of that target. Similarly, antibodies specific to a target that bind to that target and in doing so interfere with the normal biological activity of that target are inhibitors of that target, whether or not the target is an enzyme or a structural protein.

Unless particular stereoisomers are specifically indicated, all stereoisomers of the inventive compounds are included within the scope of the invention, as pure compounds as well as mixtures thereof. Unless otherwise indicated, individual enantiomers, diastereomers, geometrical isomers, and combinations and mixtures thereof are all encompassed by the present invention. Polymorphic crystalline forms and solvates are also encompassed within the scope of this invention.

Protected forms of the inventive compounds are included within the scope of this invention. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety.

The present invention includes within its scope prodrugs of the compounds of this invention. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in “Design of Prodrugs,” H. Bundgaard ed., Elsevier, 1985.

In one aspect of the invention, novel cytotoxin-targeting molecule conjugates are provided of the formula

T—L—C

Wherein T is a targeting molecule, L is a pH-sensitive or redox potential-sensitive linker, and C is a cytotoxin. Said conjugates are prepared by initial coupling of linker L to cytotoxin C, then coupling the L—C so produced to targeting molecule T so as to provide T—L—C. Alternatively, linker L is first coupled to targeting molecule T, then coupling the T—L so produced to cytotoxin C so as to provide T—L—C. Methods for coupling linkers and linker-cytotoxin pairs to targeting molecules such as antibodies, growth factors, and polysaccharides are described for use in other applications in F. Kratz et al., “Drug-Polymer Conjugates Containing Acid-Cleavable Bonds,” Critical Reviews in Therapeutic Drug Carrier Systems (1999) 16: 245-288 (incorporated herein by reference). Methods to link maytansine to antibodies are described in R. J. Ravi et al., “Cytotoxic agents comprising maytansinoids and their therapeutic use,” U.S. Pat. Nos. 5,475,092 and 5,208,020 (each of which is incorporated herein by reference).

In one embodiment, the cytotoxin C is a maytansine or ansamitocin, the targeting molecule T is an antibody, growth factor, or polysaccharide, and the linker L is a pH-sensitive or redox potential-sensitive linker, wherein the ansamitocin is linked at the C9 carbon as shown in formula (I):

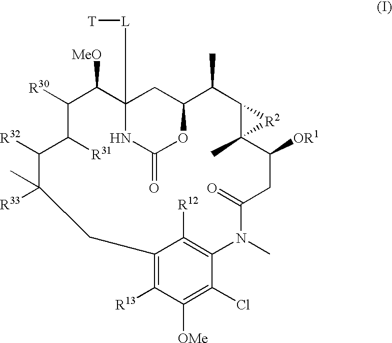

wherein

L is a pH-sensitive or redox potential-sensitive linker;

T is an antibody, growth factor, or polysaccharide;

R¹ is H, C(═O)R⁴, or C(═O)—CHMe—N(Me)—C(═O)—R⁴,

wherein R⁴ is C1-C6 straight or branched alkyl;

R² is O or a bond;

R¹² and R¹³ are each independently H, OH, or NH₂; and

R⁺is OH, R³¹ is H, and R³² and R³³ together form a bond, or R³² is OH, R³³ is H, and R³⁰ and R³¹ together form a bond.

In a preferred embodiment, the linker L is a molecule of formula

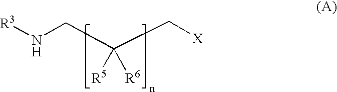

wherein R³ is connected to targeting molecule T and is selected from the group consisting of

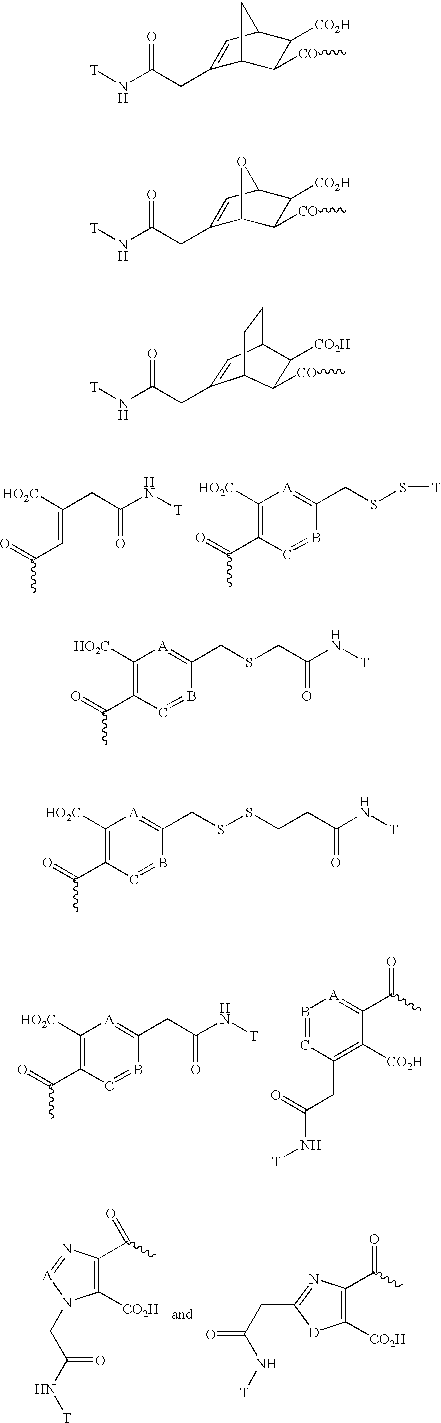

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and

D is O, NH, or S;

R⁵ and R⁶ are independently H or methyl, or taken together form ═CH₂;

X is connected to the ansamitocin and is O, NH, S, O—(C═O)—O, S—(C═O)—O, O—(C═O)—S, S—(C═O)—S, O—(C═O)—NH, S—(C═O)—NH, or NH—(C═O)—NH; and

n=0, 1, or 2.

In these molecules, the targeting molecule T is coupled to the linker L through an amide bond or a disulfide bond as indicated in the definition of group R³. This embodiment provides conjugates linked using pH-sensitive linkers.

In a second embodiment, the linker L in formula (I) is a molecule of formula (B)

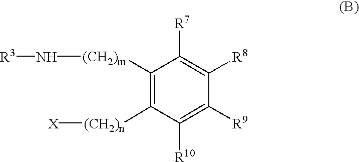

wherein

R³ is connected to targeting molecule T and is selected from the group consisting of

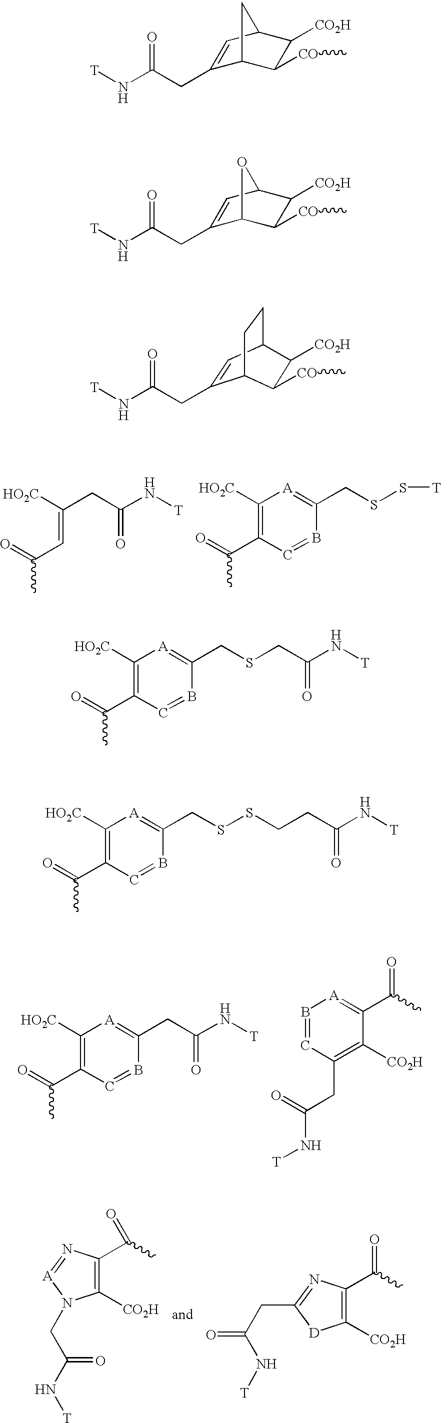

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and

D is O, NH, or S;

R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;

X is connected to the ansamitocin and is O, NH, or S; and

m and n are each independently 0, 1, 2, or 3.

In these molecules, the targeting molecule T is coupled to the linker L through an amide bond or a disulfide bond as indicated in the definition of group R³. This embodiment provides conjugates linked using pH-sensitive linkers.

In a third embodiment, the linker L in formula (I) is a molecule of formula (C)

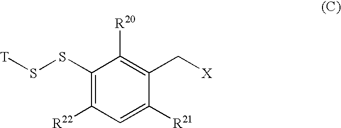

wherein

R²⁰, R²⁰, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl; and

X is connected to the ansamitocin and is O, S, or NH.

In these molecules, the targeting molecule T is coupled to the linker L through an amide bond or a disulfide bond as indicated in the definition of group R.

In other embodiments of the invention, ansamitocin-targeting molecule conjugates are provided wherein the ansamitocin is linked at the 3-hydroxyl group as shown in formula (II):

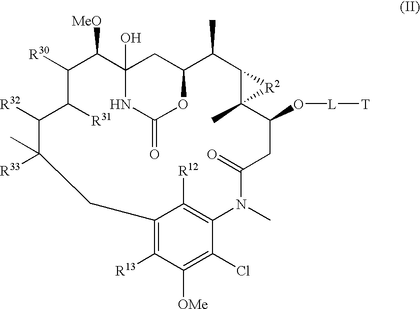

wherein

L is a pH-sensitive or redox potential-sensitive linker;

T is an antibody, growth factor, or polysaccharide;

R² is O or a bond;

R¹² and R¹³ are each independently H, OH, or NH₂; and

R³⁰ is OH, R³¹ is H, and R³² and R³³ together form a bond, or R³² is OH, R³³ is H, and R³⁰ and R³¹ together form a bond.

In one embodiment, the linker L in formula (II) is a molecule of formula (D)

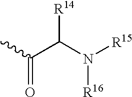

wherein

R¹⁴ is H or methyl;

R¹⁵ is H or methyl; and

R¹⁶ is taken from the group consisting of

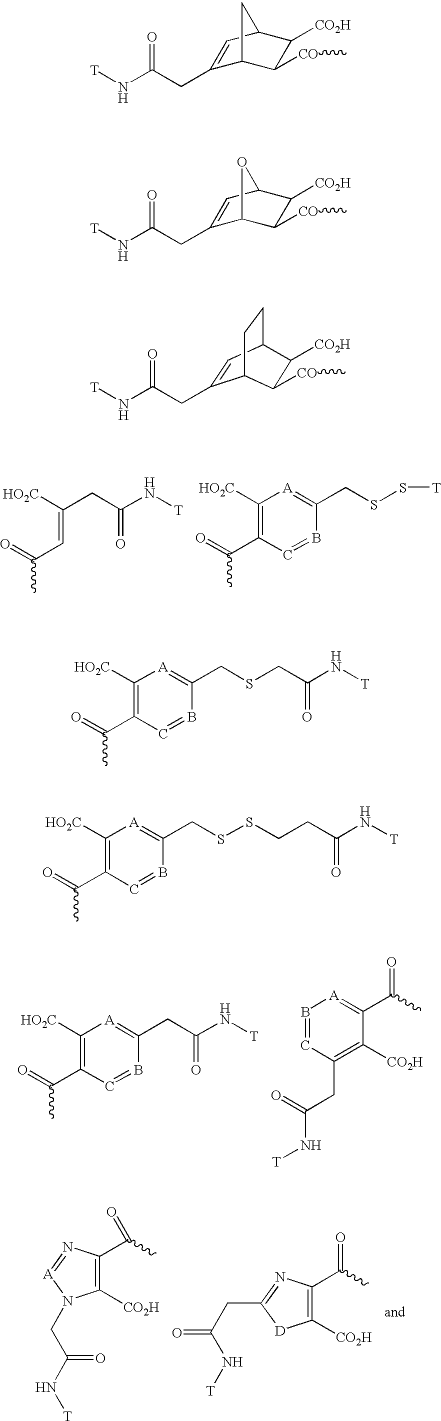

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl;

D is O, NH, or S; and

R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl.

In these molecules, the targeting molecule T is coupled to the linker L through an amide bond or a disulfide bond as indicated in the definition of group R¹⁶.

In other embodiments, multi-valent ansamitocin-targeting molecule conjugates of the formula

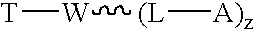

are provided wherein

T is a targeting molecule;

W is a multivalent spacer of valency z;

L is a pH-sensitive or redox potential-sensitive linker;

A is an ansamitocin; and

z is from 1 to 128.

These conjugates provide a means of increasing the number of cytotoxins delivered to a cell or tissue by the targeting molecule, thus increasing the potency of the conjugate.

In one embodiment, the multi-valent ansamitocin-targeting molecule conjugate has the formula (III)

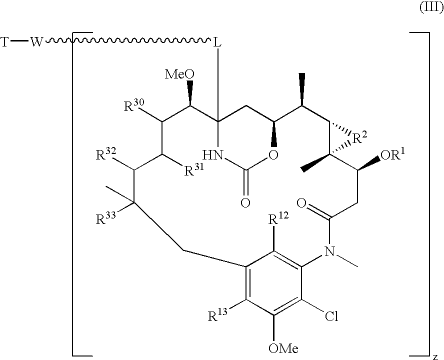

wherein

L is a pH-sensitive or redox potential-sensitive linker;

W is a multivalent dendrimer of valency=z;

T is an antibody, growth factor, or polysaccharide

R¹ is H, C(═O)R⁴, or C(═O)—CHMe—N(Me)—C(═O)—R⁴,

wherein R⁴ is C1-C6 straight or branched alkyl;

R² is O or a bond;

R¹² and R¹³ are each independently H, OH, or NH₂;

R³⁰ is OH, R³¹ is H, and R³² and R³³ together fonn a bond, or R³² is OH, R³³ is H, and R³⁰ and R³¹ together form a bond; and

z is from 1 to 128.

In a preferred embodiment, the linker L in formula (III) is a molecule of formula (A)

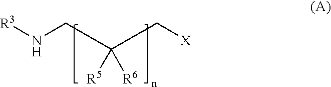

wherein R³ is connected to one valency of dendrimer W and is selected from the group consisting of

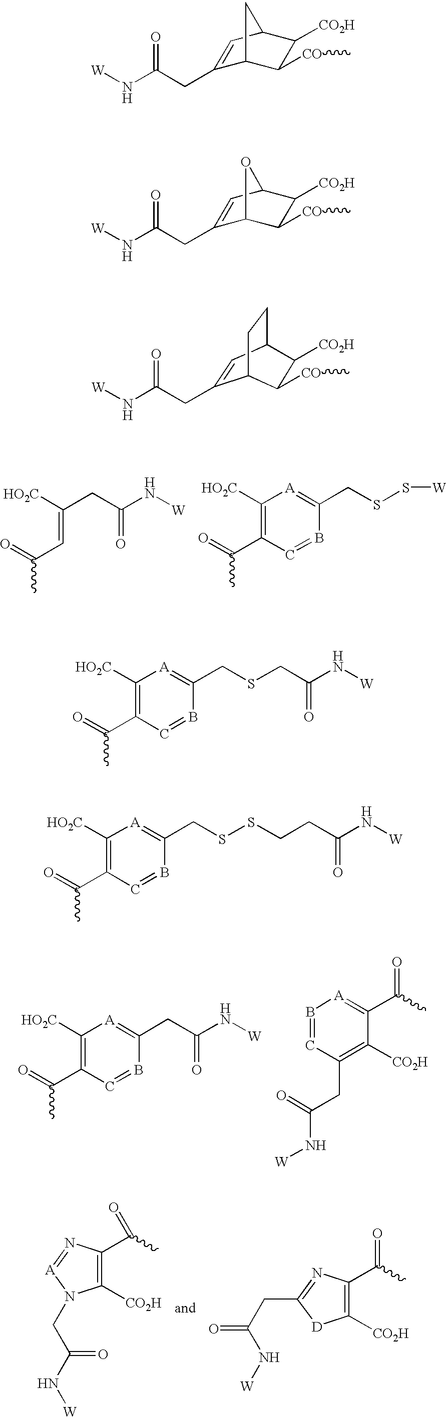

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and

D is O, NH, or S;

R⁵ and R⁶ are independently H or methyl, or taken together form ═CH₂;

X is connected to the ansamitocin and is O, NH, S, O—(C═O)—O, S—(C═O)—O, O—(C═O)—S, S—(C═O)—S, O—(C═O)—NH, S—(C═O)—NH, or NH—(C═O)—NH; and

n=0, 1, 2, or 3.

The dendrimer W is connected to the linker L through an amide or disulfide bond as indicated in the above definition of R³. The dendrimer W is also connected to the targeting molecule T through an amide or disulfide bond to a remaining valency of dendrimer W.

In another embodiment, the linker L in formula (III) is a molecule of formula (B)

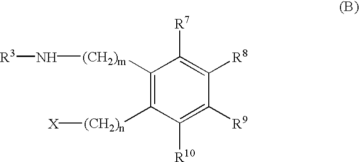

wherein R³ is connected to one valence of multivalent spacer W and is selected from the group consisting of

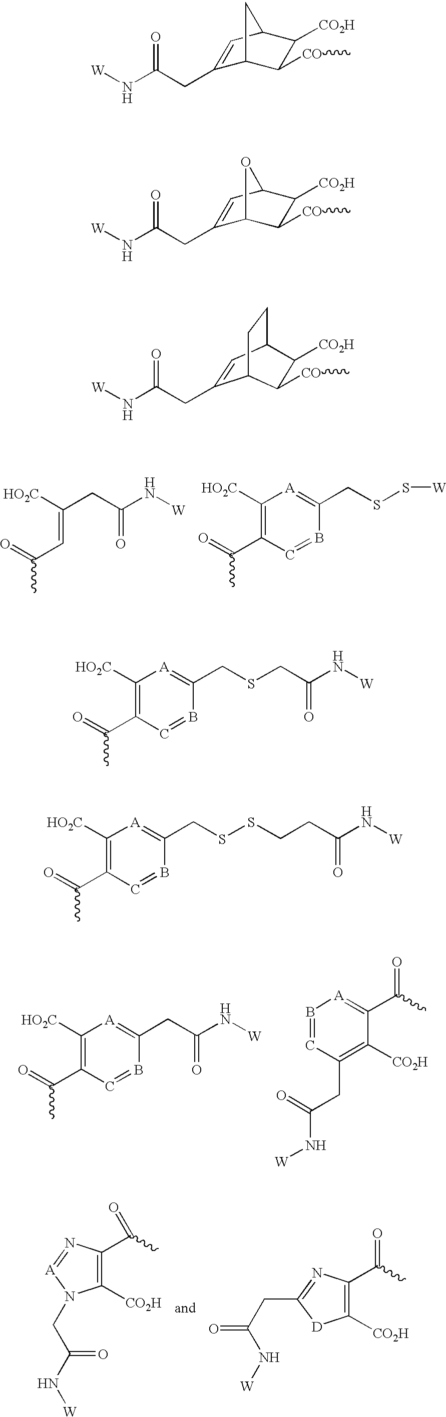

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and

D is O, NH, or S;

R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;

X is connected to the ansamitocin and is O, NH, or S; and

m and n are each independently 0, 1, 2, or 3.

The dendrimer W is connected to the linker L through an amide or disulfide bond as indicated in the above definition of R³. The dendrimer W is also connected to the targeting molecule T through an amide or disulfide bond to a remaining valency of dendrimer W.

In another embodiment, the multi-valent ansamitocin-targeting molecule conjugate has the formula (IV)

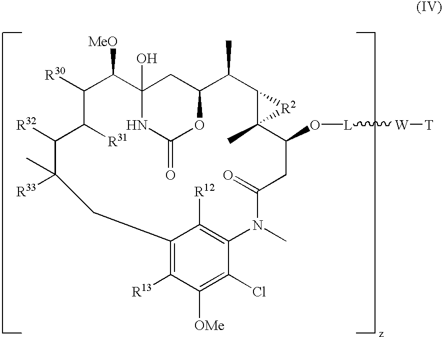

wherein

L is a pH-sensitive or redox potential-sensitive linker;

W is a multivalent spacer of valency=z;

T is a targeting molecule;

R² is O or a bond;

R¹² and R¹³ are each independently H, OH, or NH₂;

R³⁰ is OH, R³¹ is H, and R³² and R³³ together form a bond, or R³² is OH, R³³ is H, and R³⁰ and R³¹ together form a bond; and

z is from 1 to 128.

In one embodiment, the linker L in formula (IV) is a molecule of formula (D)

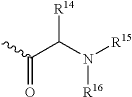

wherein

R¹⁴ is H or methyl;

R¹⁵ is H or methyl; and

R¹⁶ is connected to one valence of multivalent spacer W and is taken from the group consisting of

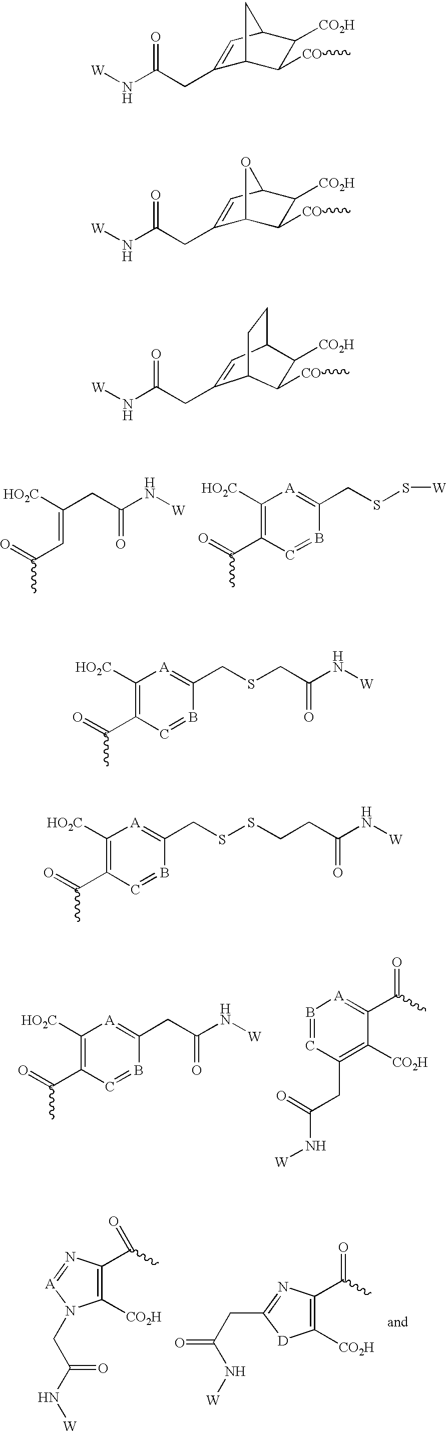

wherein A, B, and C are each independently N or CR¹⁹,

wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl;

D is O, NH, or S; and

R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl.

The dendrimer W is connected to the linker L through an amide or disulfide bond as indicated in the above definition of R¹⁶. The dendrimer W is also connected to the targeting molecule T through an amide or disulfide bond to a remaining valency of dendrimer W

In all the above embodiments, the multivalent spacer W is a polylysine dendrimer, StarBurst dendrimer, or similar multi-valent functionalized dendrimer. The dendrimers can be polyamines, polycarboxylates, or polythiols. The valency of the dendrimer W is between 1 and 128. The targeting molecule T is an antibody, growth factor, or polysaccharide connected to the dendrimer through one valency.

In one embodiment, the ansamitocins are linked to the targeting molecule by formation of an amide bond between a carboxylate group on the linker and an amino group on the targeting molecule. In a preferred embodiment, the amino group on the targeting molecule is on a glycosidic residue of the targeting molecule. Such carbohydrate amino groups are introduced by periodiate oxidation of cis-diols followed by reductive amination with amines or diamines. In another preferred embodiment, the amino group on the targeting molecule is an ε-amino group of a lysine residue or the α-amino group of the first polypeptide residue.

In another embodiment, the ansamitocins are linked to the targeting molecule by formation of a disulfide linkage. In a preferred embodiment, the disulfide is formed using a thiol group on the targeting molecule that has been introduced via chemical derivatization of a lysine amino group with a mercaptoalkanoic acid, for example through formation of a lysine ε-(mercaptoalkanoyl)amide. In another preferred embodiment, the disulfide is formed using a thiol group on the targeting molecule that has been introduced via chemical derivatization of a glycosidic residue on the targeting molecule, for example through periodate oxidation of a cisdiol followed by reductive amination using an aminoalkylthiol. In a preferred embodiment, the reactivity of the disulfide linkage is selectable through the use of the appropriate linking moiety.

In another embodiment, the ansamitocins are linked to a targeting molecule by formation of a sulfenamide linkage. In a preferred embodiment, the sulfenamide is formed using a thiol group on the targeting molecule that has been introduced via chemical derivatization of a lysine amino group with a mercaptoalkanoic acid. In another preferred embodiment, the sulfenamide is formed using a thiol group on the targeting molecule that has been introduced via chemical derivatization of a glycosidic residue on the targeting molecule.

In another aspect of the invention, novel ansamitocins having hydroxyl groups at the 11, 13, 17, and/or 21-positions are prepared by genetic engineering of the ansamitocin polyketide synthase (PKS). In one embodiment, 11-hydroxyansamitocins, 13-hydroxyansamitocins, and 11,13-dihydroxyansamitocins are prepared by inactivation of dehydratase domains within the PKS. The dehydratase activities are inactivated either by deletion, by mutagenesis, by replacement of the entire reduction domain with a heterologous domain naturally lacking a dehydratase activity, or by replacement of the entire module with a heterologous module naturally lacking a dehydratase activity. Methods for performing such inactivations are described in McDaniel, “Library of Novel “Unnatural” Natural Products,” PCT publication 00/024907; Gokhale et al., “Methods to Mediate PKS Module Effectiveness,” PCT publication 00/47724 (each of which is incorporated herein by reference). In another embodiment, 17-hydroxyansamitocins, 21-hydroxyansamitocins, 17,21-dihydroxyansamitocins, 17-aminoansamitocins, 21-aminoansamitocins, 17,21-diaminoansamitocins, 17-hydroxy-21-aminoansamitocins, and 17-amino-21-hydroxyansamitocins are prepared by feeding substituted benzoic acids to an organism expressing the ansamitocin PKS which is incapable of normal production of the ansamitocin starter unit. The use of mutants of organisms defective in starter unit production, which in wild-type form normally produce polyketides, for the preparation of polyketides through acid feeding has been described in Dutton et al., “Novel Avermectins Produced by Mutational Biosynthesis,” J. Antibiotics, (1991) 44: 357-365; and Gibson et al., “Antiparasitic Agents,” U.S. Pat. No. 5,089,480 (each of which is incorporated herein by reference).

In another aspect of the invention, the novel ansamitocins prepared by genetic engineering of the ansamitocin PKS are linked to a targeting molecule by formation of a linkage to the novel hydroxyl or amino groups, using one of the pH-sensitive or redox potential-sensitive linkers described above via ester or amide linkages.

In another aspect of the invention, disulfide linkages of varying reactivity towards reduction are provided. In one embodiment, the disulfide linkage comprises a mixed alkyl-aryl disulfide wherein the aryl moiety is substituted by alkyl and electron-withdrawing groups so as to allow variation in the susceptibility of the disulfide towards thiol-disulfide interchange. Thus, compounds of formula (V) are provided

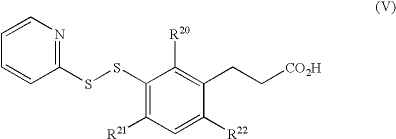

wherein R²⁰, R²¹, and R²² are each independently straight or branched C₁-C₄ alkyl, C₁-C₄ alkoxy, halogen, amino, alkylamino, dialkylamino, nitro, formyl, carboxyl, or alkoxycarbonyl.

In another embodiment, the compounds of formula (V) are used to link a cytotoxin to a targeting molecule. In a preferred embodiment, the cytotoxin is a thiol-containing ansamitocin, and the targeting molecule is an antibody, growth factor, or polysaccharide directed against a cancer cell. The targeting molecule is connected to the compound of formula (V) through an amide bond formed between the carboxyl group of (V) and an amino group of the targeting molecule, said amino group being part of either an amino acid side chain or a glycosidic residue.

In another preferred embodiment, the cytotoxin is an ansamitocin wherein the carboxyl moiety of compound (V) replaces the acetyl moiety on the C3-side chain, and the targeting molecule is a thiol-containing antibody, growth factor, or polysaccharide directed against a cancer cell. The thiol on the targeting molecule may be either a cysteine side chain or may be a thiol introduced through chemical derivatization, for example to a glycosidic residue.

In another aspect of the invention, the novel ansamitocins are linked to a targeting molecule through a dendritic linker or connector so as to increase the number of maytansine molecules delivered by the targeting molecule. In one embodiment, the dendritic linker or connector is a Starburst® dendrimer having at least four, preferably at least eight, and more preferably at least sixteen surface amine groups. In another embodiment, the dendritic linker or connector is a polylysine formed by generating amide bonds to both the α- and ε-amino groups of lysine. In another embodiment, the polylysine dendrimer is first treated with ethylene episulfide in order to produce a dendrimer having multiple surface thiol groups. In a preferred embodiment, the dendrimer is transiently linked to both the targeting molecule and the maytansine analog through pH-sensitive or redox potential-sensitive linkers. In another embodiment, the dendrimer comprises a disulfide group suitable for attachment of the targeting molecule and an array of other functionality suitable for attachment of multiple cytotoxins. Preparation of such dendrimers for other purposes is described in Klimash et al., “Disulfide-containing dendritic polymers,” U.S. Pat. No. 6,020,457 (incorporated herein by reference).

In preferred embodiments of the invention, the targeting molecule used to target the ansamitocin analog is directed against a cancer cell. In preferred embodiments, the targeting molecule is an antibody. In particularly preferred embodiments, the antibody is directed against a cellular receptor protein. Preferred examples include but are not limited to antibodies directed against HER2/neu, epidermal growth factor receptor (EGFR), ErbB2, platelet-derived growth factor (PDGF) receptor, vascular endothelial growth factor receptor 2 (VEGFR2 or KDR), and insulin-like growth factor receptor (IGFR). In other preferred embodiments, the antibody is directed against other clinically relevant tumor markers, including but not limited to polymorphic epithelial mucin (MUC-1), the ovarian cancer-associated antigen CA125, or against the CD33 myeloid-differentiation antigen.

In other preferred embodiments, the targeting molecule is a cellular growth factor. Preferred examples of such growth factors include but are not limited to epidermal growth factor (EGF), insulin-like growth factor (ILGF), vascular endothelial growth factor (VEGF), and platelet-derived growth factor (PDGF).

In other embodiments, the targeting molecule is a polysaccharide ligand for a cellular receptor. Preferred embodiments include but are not limited to ligands for the selectin receptors, such as Lewis-x, and ligands for growth factor receptors. Examples of polysaccharide ligands that are ligands for growth factor receptors are described in J. L. Magnani and E. G. Bremer, “Pharmaceutical compositions for treatment of EGF receptor associated cancers,” U.S. Pat. No. 6,281,202, and J. L. Magnani and E. G. Bremer, “Methods for treatment of EGF receptor associated cancers” U.S. Pat. No. 6,008,203 (each of which is incorporated herein by reference).

The described pH-sensitive linkers based upon cis-aconitic acid are limited to linking the cytotoxin to the targeting molecule through amide and/or ester bonds. In the present invention, conjugation of the cytotoxin maytansine and related analogs through an acetal-type linkage is provided. Thus, in one aspect of the invention, novel pH-sensitive linkers are provided. These linkers constitute a means of conjugating a cytotoxic agent to a targeting molecule, where the cytotoxic agent is released from the conjugate upon uptake into a cellular compartment of sufficiently low pH.

The use of acetal glycoside linkages to form prodrugs of DNA alkylating agents was reported by Tietze et al., “Development of custom-made, acid-catalytically activatable cytostatics for selective tumor therapy,” Angew. Chem. Int. Ed. Eng. (1990) 29: 782. Acetal-based cross linkers have been developed to make antibody-diphtheria toxin conjugates (e.g., Srinivasachar & Neville, “New protein cross-linking reagents that are cleaved by mild acid,” Biochemistry (1989) 28: 2501, incorporated herein by reference). The relatively slow cleavage of acetals at pH values above 4 has hindered their development as pH-sensitive linkers, however. In one aspect of the present invention, novel acetal-based linkers having enhanced pH-sensitivity due to the incorporation of an intramolecular acid catalyst are provided. These novel linkers are used either with known cytotoxins or with novel cytotoxins of the invention.

In one embodiment, the acid-sensitive linker is a bicyclic endo-dicarboxylic acid derivative. As shown in Scheme 1, these linkers may be prepared by the reaction of a bicyclic endo-dicarboxylic acid anhydride with an aminoalcohol. The bicyclic endo-dicarboxylic acid anhydrides may be readily prepared through a Diels-Alder reaction of 3-furanacetic acid or 3-cyclopentadieneacetic acid with maleic anhydride.

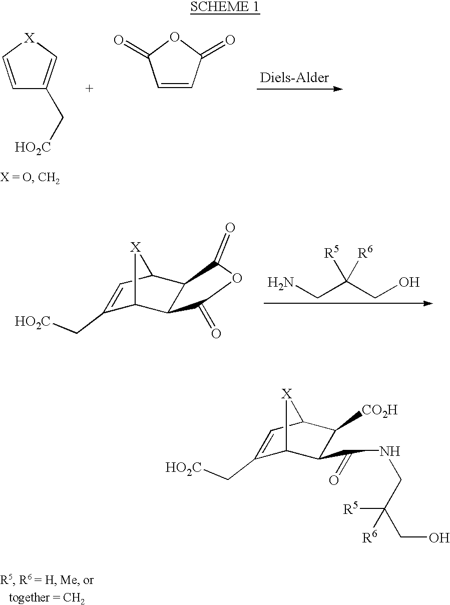

Similar linkers can be prepared using other bicyclic systems, for example bicyclo[2.2.2]octenes prepared by Diels-Alder reaction of maleic anhydride with cyclohexadiene-acetic acid.

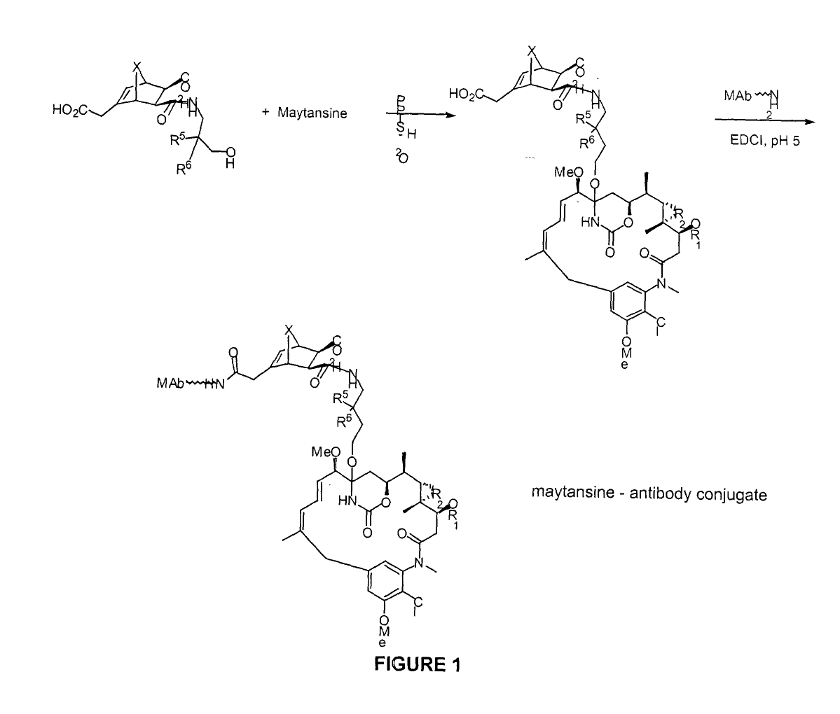

The resulting linker is attached to the cytotoxin using an acetal-type linkage. For example, with maytansine and ansamitocin analogs, the aminoalcohol is reacted with maytansine until mildly acidic anhydrous conditions so as to form the acetal at C9 (FIG. 1). The linked maytansine is then conjugated to the targeting molecule through an amide linkage, for example using a water-soluble carbodiimide coupling with EDCI (1-[3-(dimethylamino)-propyl]-3-ethylcarbodiimide hydrochloride).

The endo-dicarboxylic acid is designed to provide an intramolecular acid catalyst that accelerates the hydrolysis of the amide linkage at low pH. This hydrolysis liberates an ammonium group that subsequently acts as a second intramolecular acid catalyst to accelerate the hydrolysis of the acetal linkage to the cytotoxin. The use of the ammonium group to catalyse acetal hydrolysis thus overcomes the difficulty of slow acetal hydrolysis at relevant pH values.

The aminoalcohol component of the linker facilitates hydrolysis of the acetal once the protonated amine group is available. In a preferred embodiment, the aminoalcohol is a 1,3-aminopropane, optionally substituted with methyl, a gem-dimethyl, or methylene groups. The presence of methyl substituents accelerates the acetal hydrolysis by enforcing a conformation in which the ammonium group is positioned adjacent to the acetal oxygen, thus improving the general acid catalysis by the ammonium group. The presence of a methylene serves to lower the pKₐ of the allylic ammonium group, thus increasing the acidity of the group and accelerating the rate of hydrolysis. In other embodiments, the length of the carbon chain between the amine and the alcohol and the number and positioning of methyl and/or methylene substituents is varied so as to optimize the hydrolysis reaction.C

In other embodiments, the acetal-linked cytotoxin is conjugated to the targeting molecule through a disulfide linkage. The appropriate linker is prepared according to the methods of the invention as shown in Scheme 2.

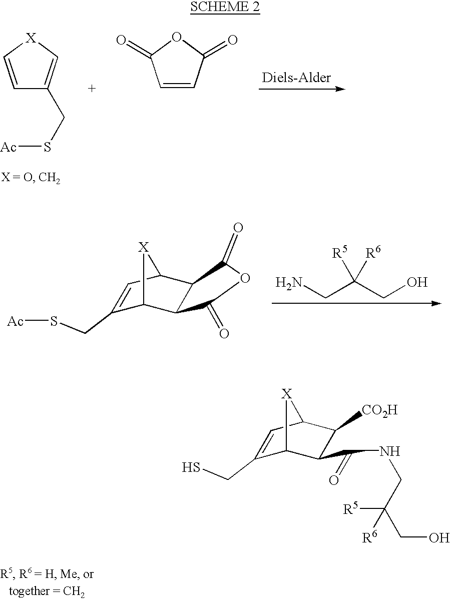

Diels-Alder reaction of 3-(acetylthio)furan or 3-(acetylthio)cyclopentadiene with maleic anhydride yields the endo-bicyclic anhydride, which is treated with the aminoalcohol described above. The thiol-protected linker is then reacted with the maytansine analog as described above. The thiol protecting group is cleaved during the amide-forming reaction, leaving a free thiol. This thiol is activated using di(2-pyridyl)disulfide to give the mixed activated disulfide. Treatment of a thiol-containing targeting molecule with the mixed activated disulfide conjugates the maytansine analog onto the targeting molecule.

In another embodiment, the thiol in the above-described linkers is replaced by an amine, which allows conjugation through a carboxyl group of a targeting molecule.

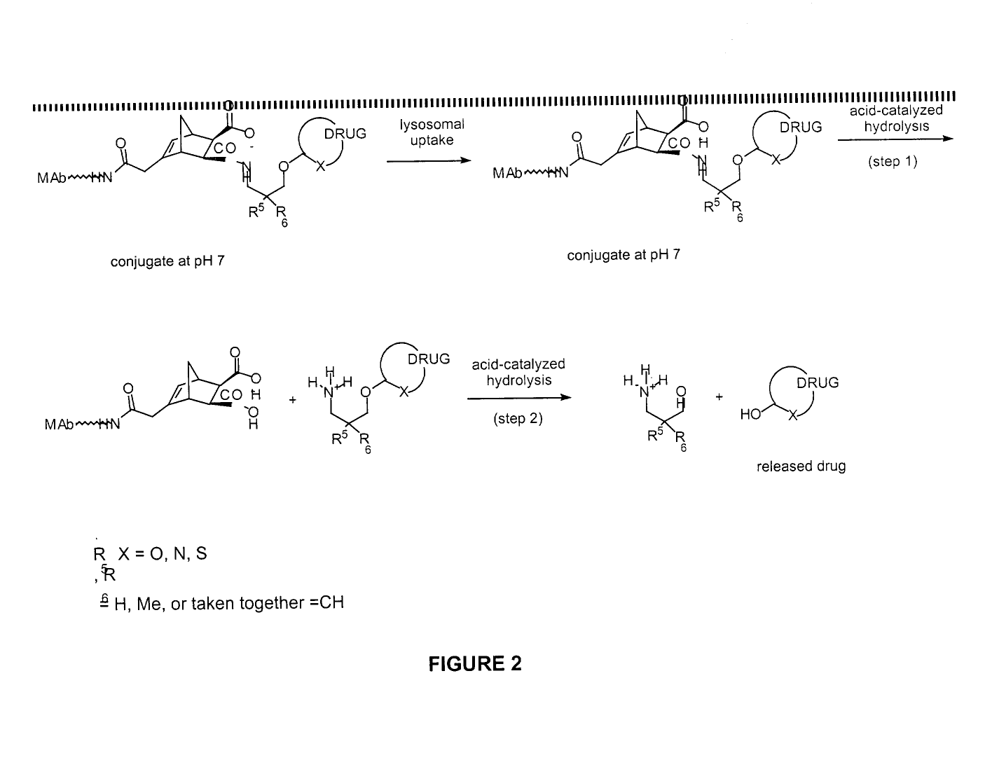

The cytotoxin conjugates illustrated by the maytansine-targeting molecule example above are hydrolyzed in the acidic environment of the lysosome. As illustrated in FIG. 2, the free carboxyl group on the linker becomes protonated at pH values below ca. 5. As this carboxyl is geometrically fixed in a cis configuration to the adjacent amide, the carboxyl acts as a general acid catalyst to accelerate the hydrolysis of the adjacent amide. This cleaves the targeting molecule and releases a pro-drug form of the maytansine analog. The liberated amino group of the acetal spacer is also completely protonated at the pH of the lysosome, and so acts to accelerate the hydrolysis of the acetal linkage at C9. Maytansine is thus freed from the conjugate in a fully active, cytotoxic form.

The present invention is a substantial improvement over the art, in that release of the cytotoxin from the conjugate requires two consecutive steps, each step requiring conditions specific to the intracellular environment. This provides an increased margin of safety. For example, if the targeting molecule-linker bond were cleaved prematurely, for example via a proteolytic enzyme when the linkage is via an amide or via disulfide exchange when the linkage is a disulfide, the released cytotoxin is still in an inactive pro-drug form. As the pH of the extracellular environment is above pH 7, release of the active cytotoxin by hydrolysis of the acetal linkage is greatly retarded unless the pro-drug has been released within the lysosome. While illustrated here using two subsequent pH-dependent steps, the two release steps can also be taken from the set of any combination of steps which can be selectively accomplished within the target cell. Such steps may include, but are not limited to, enzymatic cleavage, acid-catalyzed hydrolysis, oxidation/reduction steps, and thiol-disulfide interchange.

In another embodiment, the acid-sensitive linker is an aryl-1,2-dicarboxylic acid derivative. This linker also provides a cis-carboxyl group which acts as an acid catalyst in the pH-dependent breakdown of the conjugate as described above. Such linkers are prepared according to the invention as illustrated in Scheme 3, from the reaction of a phthalic anhydride with the aminoalcohol described above.

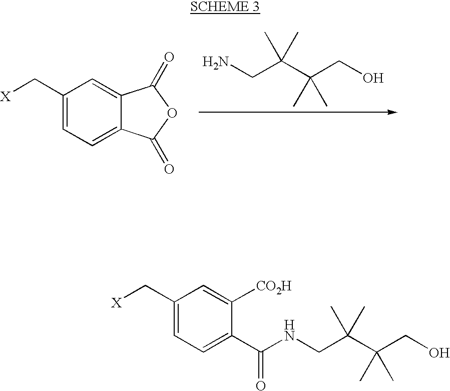

The linker is reacted with a cytotoxin, such as a maytansine analog, under mildly acidic anhydrous conditions as described above to form a C9-acetal. When X is COOH, the resulting linked maytansine is conjugated to a targeting molecule through amide formation using EDCI. When X is SAc, the thiol is deprotected during reaction with the aminoalcohol and the resulting linked maytansine is activated with di(2-pyridyl)disulfide and conjugated to a targeting molecule through a disulfide linkage.

In another embodiment, the aryl-1,2-dicarboxylic acid linker comprises a substituted heterocyclic system. Preferred examples include but are not limited to substituted pyridine-2,3-dicarboxylic acids, pyridine-3,4-dicarboxylic acids, pyrimidine-4,5-dicarboxylic acids, pyrazine-2,3-dicarboxylic acids, pyridazine-3,4-dicarboxylic acids, pyridazine-4,5-dicarboxylic acids, and the corresponding benzo-fused examples.

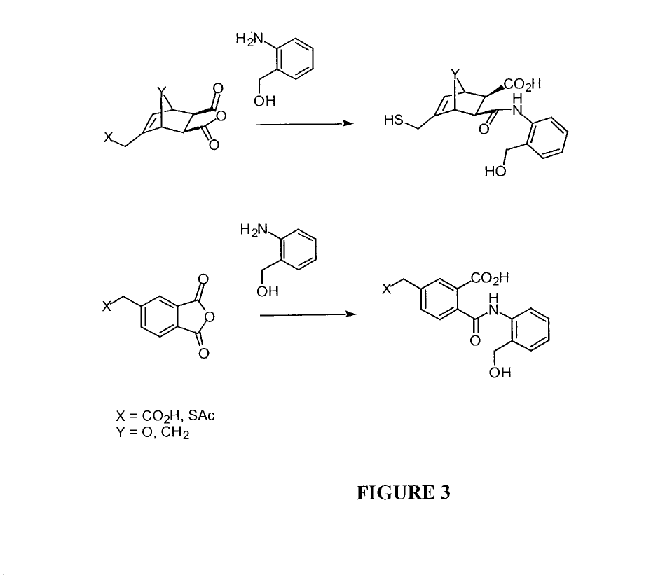

In a third embodiment, the acid-sensitive linker is a 2-aminobenzyl alcohol derivative. In this embodiment, the 2-aminobenzyl alcohol serves as a geometrically-constrained replacement for the aminoalcohol described above, and can be used as such in all the examples described above. As illustrated in FIG. 3, the linker is prepared according to the invention by reacting an anhydride with 2-aminobenzyl alcohol.

As described above, the linker is reacted with a maytansine analog under mildly acidic anhydrous conditions so as to form the C9-acetal, and then the X-group is used to conjugate to the targeting molecule. When X is COOH, the resulting linked maytansine is conjugated to a targeting molecule through amide formation using EDCI. When X is SAc, the thiol is deprotected during reaction with the aminoalcohol and the resulting linked maytansine is activated with di(2-pyridyl)disulfide and conjugated to a targeting molecule through a disulfide linkage.

In another embodiment, the 2-aminobenzyl alcohol is part of a heterocyclic system. Preferred examples include but are not limited to 2-amino-3-(hydroxymethyl)-pyridine, 3-amino-4-(hydroxymethyl)pyridine, 4-amino-5-(hydroxymethyl)-pyrimidine, 2-amino-3-(hydroxymethyl)pyrazine, and 3-amino-4-(hydroxymethyl)-pyridazine.

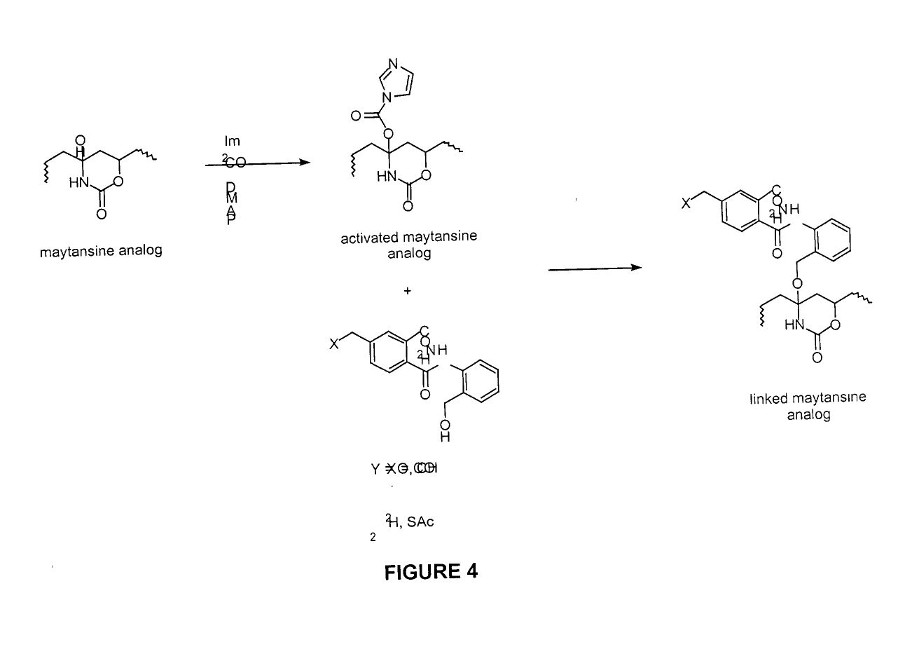

In another embodiment, the acid-sensitive linker is a 2-aminobenzyl amine derivative. These are prepared as described in FIG. 4, substituting 2-aminobenzyl amine in place of 2-aminobenzylalcohol. In another embodiment, the 2-aminobenzyl amine is part of a heterocyclic system. Preferred examples include but are not limited to 2-amino-3-(aminomethyl)pyridine, 3-amino-4-(aminomethyl)pyridine, 4-amino-5-(aminomethyl)pyrimidine, 2-amino-3-(aminomethyl)pyrazine, and 3-amino-4-(aminomethyl)pyridazine.

In another embodiment, the acid-sensitive linker is a 5-aminoacyl derivative of a linker of FIG. 4. As illustrated in FIG. 4, these linkers are prepared according to the invention by reaction of one of the above-described linkers with an alcohol moiety of the cytotoxin that has been activated as a chloroformate or imidazolide, for example using phosgene or carbonyldiimidazole, so as to form a carbonate linkage. In this embodiment, acid-catalyzed hydrolysis of the linker leads to formation of the free amino group, which cyclizes upon the carbonate and releases the active form of the cytotoxin. With the amino-aryl linkers of Scheme 3, the pKₐ of the liberated amino group is approximately 5, such that a substantial portion of the compound exists in the nucleophilic free-amine form in the acid conditions of the lysosome. In other embodiments, the aryl group is heterocyclic or is substituted by electron-donating or electron-withdrawing groups, so as to allow adjustment of the pKₐ of the liberated amino group and its nucleophilicity. This allows for tuning of the rate of decomposition of the conjugate upon reaching the lysosome.

In another aspect of the invention, novel redox potential-sensitive linkers are provided. In one embodiment, the redox potential-sensitive linker is an alkyl-aryl mixed disulfide wherein the aryl moiety is substituted so as to control the steric and electronic properties of the disulfide. Such linkers provide a means of attenuating the rate of thiol-disulfide interchange such that the linkage is stable in environments of low reductive potential, for example in the extracellular environment, but is cleaved in the presence of high concentrations of reducing thiols, for example in the intracellular environment.

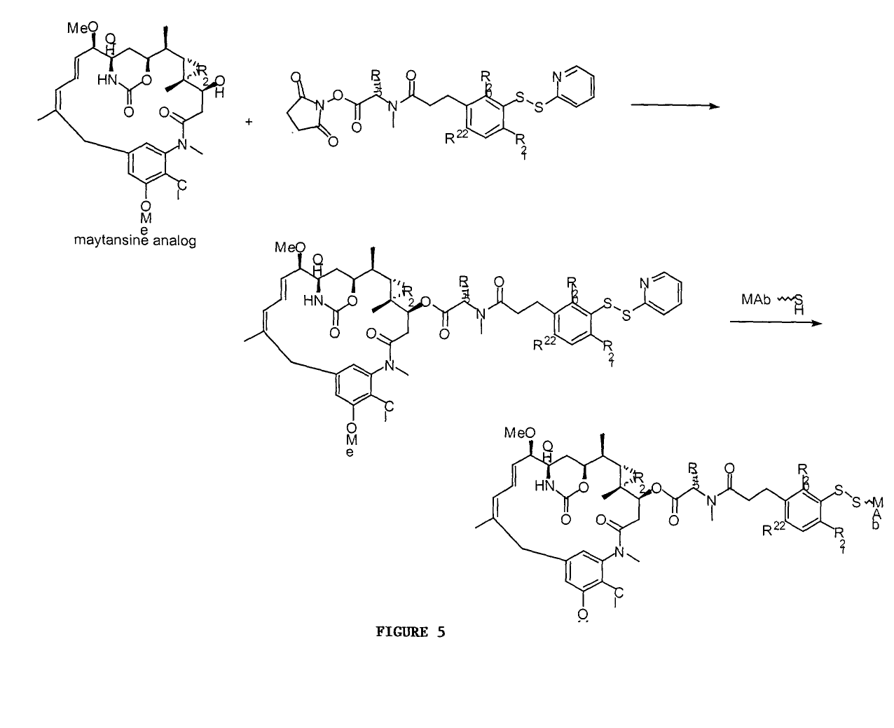

In one embodiment, the redox potential-sensitive linker is used directly to couple the targeting molecule and the cytotoxin. As an example, FIG. 5 illustrates linking maytansine to an targeting molecule via the C3-side chain. The disulfide linker comprises an arylthiol moiety, substituted with an array of sterically-demanding, electron-donating, and electron-withdrawing groups so as to allow for control of the reactivity of the disulfide in thiol-disulfide interchange reactions, and having an attached propionic acid moiety which allows for attachment to the cytotoxin via an ester or amide bond. Preferred examples of arylthiol moieties include but are not limited to 3-(3-thiophenyl)propionic acid, 3-(2,4-dimethyl-3-thiophenyl)propionic acid, 3-(4-nitro-3-thiophenyl)propionic acid, 3-(2-methyl-4-nitro-3-thiophenyl)-propionic acid, 3-(4-methoxy-3-thiophenyl)propionic acid, 3-(2,4-dimethyl-6-nitro-3-thiophenyl)propionic acid, and the like. These linkers are first activated for use (Scheme 4) by conversion of the thiol into the 2-pyridyl disulfide by treatment with di-(2-pyridyl)disulfide, then by conversion of the carboxylic acid into an active ester, for example an N-hydroxysuccinimide ester by treatment with a carbodiimide and N-hydroxysuccinimide.

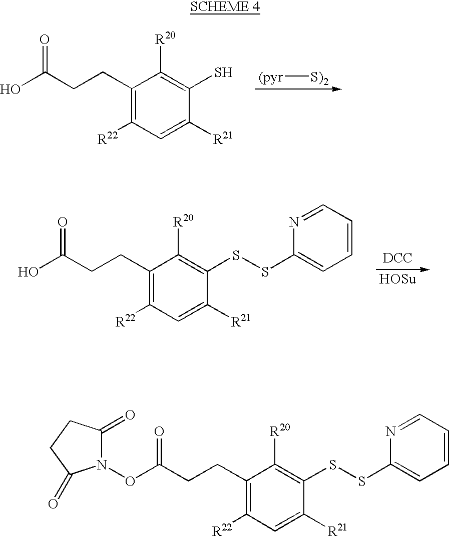

The activated linker may then be reacted with the cytotoxin followed by the thiol-containing targeting molecule. In one embodiment, the linker is used to conjugate a maytansine analog onto a targeting molecule. According to the invention, the above-described linkers are first reacted with (L)-alanine tert-butyl ester, the ester is cleaved by treatment with trifluoroacetic acid, and the alanyl-linker is again treated with a carbodiimide and N-hydroxysuccinimide. Maytansinol or a maytansinol analog is treated with the alanyl-linker to link the maytansinol, followed by reaction with a thiol-containing targeting molecule to produce the final conjugate.

In another aspect of the invention, novel maytansine analogs are produced by genetic engineering of the maytansine and/or ansamitocin biosynthetic gene clusters. The core structures of maytansine and other ansamitocins are biosynthesized by complex enzymes known as polyketide synthases.

The polyketide synthase enzymes (PKS) involved in the biosynthesis of polyketides, such as maytansine and the ansamitocins, are organized in a modular fashion, with each module containing the activities necessary for addition and processing of a 2-carbon unit onto the growing polyketide chain. The structural complexity of a polyketide arises from the large number of combinations of activities that may occur together within a module, taken together with the large number of modules within a PKS. Each module contains at least three core activities, a ketosynthase (KS), an acyltransferase (AT), and an acyl carrier protein (ACP) domain.

The AT is responsible for selection of the extender unit that is added by the module. Typical extender units are malonyl-CoA, methylmalonyl-CoA, ethylmalonyl-CoA, hydroxymalonyl-CoA, and methoxymalonyl-CoA, the use of which results in addition of a 2-carbon unit to the polyketide containing a hydrogen, methyl, ethyl, hydroxy, or methoxy substituent, respectively. The KS domain performs the addition of the extender unit, and the ACP domain functions to carry the growing chain during the catalytic cycle.

In addition to these three core activities, a module may also contain one or more modification domains that process the added 2-carbon unit. Immediately after addition by the KS, the new 2-carbon unit contains a ketone group at the 3-position. This ketone may be reduced to an alcohol by a ketoreductase (KR) domain. The resulting alcohol may be eliminated to form a 2,3-alkene by a dehydratase (DH) domain. The 2,3-alkene may be further reduced to an alkane by the action of an enoylreductase (ER) domain. Other modifications to the added extender unit, such as methylation, may also be performed if the appropriate modification domains are present.

Replacement of a domain can be used to alter the product of the module. Thus, replacement of an acyltransferase (AT) domain of a module with one from a different module having a different extender unit specificity results in a module having an altered specificity for the extender unit. It is thus possible to replace a methyl substituent in a polyketide with, for example, a hydrogen or ethyl group. Similarly, replacement of the set of reductive cycle processing domains (KR, DH, and ER) in a module with a different set results in a module having a different reductive cycle outcome. In this way, a carbon-carbon double bond in a polyketide can be replaced by an alcohol or ketone, or an alcohol can be replaced by a hydrogen. Methods for performing domain substitutions within a PKS have been described by McDaniel et al. (1997) “Gain-of-function mutagenesis of a modular polyketide synthase,” J. Am. Chem. Soc. 119:4309-4310, Liu et al. (1997) “Biosynthesis of 2-nor-6-deoxyerythronolide B by rationally designed domain substitution,” J. Am. Chem. Soc. 119:10553-10554, and Xue and Santi, “Multi-plasmid approach to preparing large libraries of polyketides,” PCT publication 00/63361, McDaniel, “Library of Novel “Unnatural” Natural Products, PCT publication 00/24907, Khosla et al., “Modular PKS Gene Cluster as Scaffold,” PCT publication 98/49315, and Khosla et al., “Recombinant production of novel polyketides,” U.S. Pat. No. 5,962,290 (each of which is incorporated herein by reference).

The maytansine and/or ansamitocin PKS genes are cloned from a producing strain and manipulated as described above to make maytansine analogs. Methods for the cloning of PKS genes have been described by Santi et al., “Method for Cloning PKS Genes,” PCT publication 01/53533 (incorporated herein by reference).

The maytansine and/or ansamitocin gene cluster or mutated versions of the gene cluster prepared according to the methods of the invention can be expressed either in the native producing organism or in host cells other than the native producer. Methods for the heterologous expression of PKS gene clusters have also been described, both in Escherichia coli (Santi et al., “Heterologous Production of Polyketides,” PCT publication 01/31035), yeasts (Barr et al., “Production of Polyketides in bacteria and Yeasts,” U.S. Pat. Nos. 6,033,883 and 6,258,566; PCT publication 98/27203), and actinomycetes (Khosla et al., “Recombinant Production of Novel Polyketides,” U.S. Pat. Nos. 5,672,491, and 5,843,718), each of which is incorporated herein by reference. Host cells suitable for heterologous expression of PKS genes are described, for example, in Khosla et al., “Recombinant Production of Novel Polyketides,” U.S. Pat. No. 5,830,750 (incorporated herein by reference).

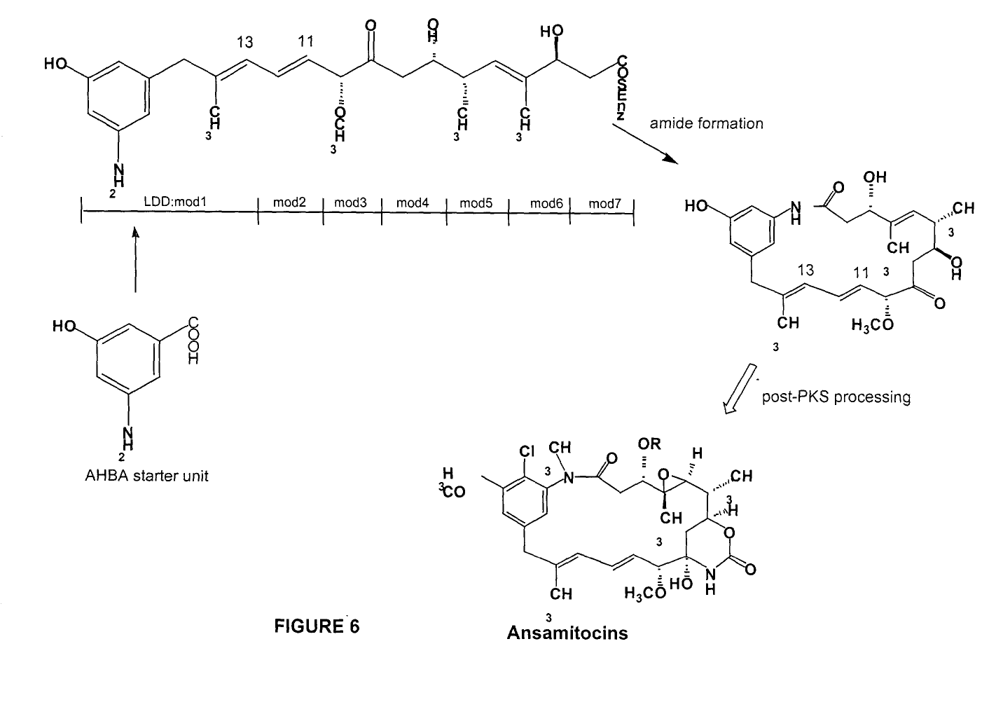

In one embodiment, 11-hydroxymaytansine analogs and 13-hydroxymaytansine analogs are provided by inactivation of the dehydratase activity in module 3 or module 2, respectively, of the maytansine and/or ansamitocin polyketide synthase. The anticipated organization of the ansamitocin PKS is shown in FIG. 6. The dehydratase activities are inactivated either by directed mutagenesis of the dehydratase domain, by deletion of the dehydratase domain, by replacement of the reductive domain of the module with a reductive domain comprising only a ketoreductase, or by replacement of the entire module with a module lacking a dehydratase domain.

Inactivation of dehydratase domains in accord with the methods of the present invention may also be obtained through random mutagenesis of the organism that normally produces maytansine and/or ansamitocins. Spores of the producing organism are either treated with a chemical mutagen, for example 1-methyl-3-nitro-1-nitrosoguanidine (MNNG), dimethylsulfate, or the like, or with mutagenic levels of radiation, for example ultraviolet radiation. The surviving spores are then allowed to grow on a suitable medium, and the resulting cultures are analyzed, for example by LC-mass spectrometry, for the presence of the desired new maytansine analog. Methods for the random mutagenesis of actinomycetes are described in Kieser et al, “Practical Streptomyces Genetics,” The John Innes Foundation, Norwich (2000) (incorporated herein by reference).

In another embodiment of the invention, novel maytansine analogs having hydroxyl or amino groups at C17, C18, C19, or C21 are provided by supplying the appropriate starter unit analog to a culture of a mutant of the producing organism deficient in production of the natural starter unit. Disruption of natural starter unit production is accomplished according to the present invention either by mutational inactivation of a gene involved in starter unit production, through mutational inactivation of the first KS domain of the maytansine and/or ansamitocin PKS, or through heterologous expression of the biosynthetic genes in a host cell that is naturally deficient in starter unit production.

The maytansines and ansamitocins contain an aromatic moiety known as the “mC₇N” unit. This mC₇N arises from incorporation of 3-amino-5-hydroxybenzoate (AHBA) as the starter unit for polyketide synthesis. AHBA is thought to arise from a brach of the shikimate pathway in which phosphoenol pyruvate is condensed with erythritol-4-phosphate in the presence of an ammonia source to provide 3,4-dideoxy-4-amino-D-arabino-heptulosonate 7-phosphate is the first committed intermediate (Müller et al., “Synthesis of (−)-3-amino-3-deoxyquinic acid,” J. Org. Chem. (1998) 63: 9753-9755, incorporated herein by reference). Several enzymes are unique to the AHBA pathway, for example those involved in the production of 3,4-dideoxy-4-amino-D-arabino-heptulosonate 7-phosphate, 5-deoxy-5-amino-3-dehydroquinic acid, 5-deoxy-5-amino-3-dehydroshikimic acid, and 5-deoxy-5-aminoshikimic acid, and inactivation of any of these enzymes will result in a deficiency in maytansine and ansamitocin starter unit biosynthesis.

In one embodiment of the invention, one or more enzymes involved in AHBA biosynthesis is inactivated. This inactivation can either be through random mutagenesis followed by screening to isolate the desired phenotype, or can be the result of directed mutagenesis. Methods for random mutagenesis are well known in the art as described above. Methods for directed mutagenesis are also well known in the art, for example by gene disruption and by gene replacement as described in Kieser et al, “Practical Streptomyces Genetics,” The John Innes Foundation, Norwich (2000) (incorporated herein by reference). A culture of the mutant organism is supplied with a suitable starter unit, for example 3-amino-5-hydroxybenzoate (AHBA), 3-amino-2,5-dihydroxy-benzoate, 3-amino-5,6-dihydroxybenzoate, 2,3-diamino-5-hydroxybenzoate, 2,6-diamino-5-hydroxybenzoate, and the like, in order to prepare the corresponding hydroxylated and/or amino ansamitocin analogs.

In another embodiment of the invention, a host cell that is naturally unable to produce AHBA is used for heterologous expression of the maytansine and/or ansamitocin PKS genes. Suitable host cells include but are not limited to Streptomyces coelicolor, Streptomyces lividans, Saccharopolyspora erythraea, Streptomyces fradiae, Myxococcus xanthus, Escherichia coli, and Saccharomyces cerevesiae. Methods for heterologous expression of PKS genes have been described above. When expressed in such a host, polyketide production is dependent upon supplying the host culture with an appropriate starter unit. A culture of the mutant organism is supplied with an analog of AHBA as described above in order to prepare the corresponding ansamitocin analogs.

In another embodiment of the invention, the first KS domain of the maytansine and/or ansamitocin PKS is inactivated. The resulting enzyme is unable to produce the maytansine or ansamitocin polyketide, unless supplied with a thioester which comprises the product of the first module of the PKS, or an analog. This technology is described in Khosla et al., U.S. Pat. Nos. 6,066,721, 6,261,816, 6,080,555, and 6,274,560 (each of which is incorporated herein by reference). A culture of the mutant organism is supplied with an analog of the module 1 product as a thioester, for example 3-(3-amino-5-hydroxyphenyl)-2-methylpropionate N-acylcysteamine thioester, 3-(3-amino-2,5-dihydroxyphenyl)-2-methylpropionate N acylcysteamine thioester, 3-(3-amino-5,6-dihydroxyphenyl)-2-methylpropionate N-acylcysteamine thioester, 3-(3,6-diamino-5-hydroxyphenyl)-2-methylpropionate N-acylcysteamine thioester, 3-(2,3-diamino-5-hydroxyphenyl)-2-methylpropionate N-acylcysteamine thioester, and the like, in order to prepare the corresponding ansamitocin analogs. Methods of preparing N-acylcysteamine thioesters are described in Ashley et al., “Synthesis of Oligoketides,” PCT publication 00/44717, incorporated herein by reference.

The polyketide synthase produces the polyketide component of the ansamitocin. Subsequent tailoring enzymes are responsible for functionalization of the polyketide, for example addition of ester linkages, epoxides, halogenation, methylation, and the like. Thus, the engineered polyketide synthases of the invention provide novel polyketides of the formula (VI)

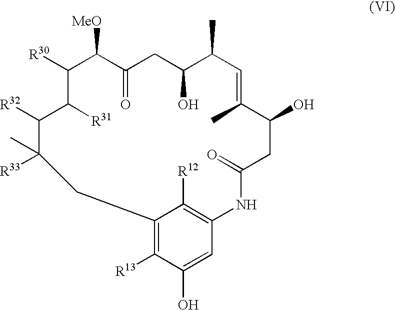

wherein R¹² and R¹³ are each independently H, OH, or NH₂; and R³⁰ is OH, R³¹ is H, and R³² and R³³ together form a bond, or R³² is OH, R³³ is H, and R³⁰ and R³¹ together form a bond, or R³⁰ and R³¹ together form a bond and R³² and R³³ together form a bond; with the proviso that when R³⁰ and R³¹ form a bond and R³² and R³³ form a bond, that either R¹² or R¹³ cannot be H.

When produced in an organism lacking the subsequent tailoring enzymes, the polyketides of formula (VI) can be isolated and subjected to further chemical modification. Alternately, the polyketides of formula (VI) can be produced in a host cell containing a subset of the tailoring enzymes, so as to produce novel modified analogs. Such host cells containing a subset of the tailoring enzymes can be prepared either by mutagenesis of the natural producing organism, so as to remove one or more enzymes from the host cell, or by addition of subsets of the genes encoding the tailoring enzymes into a heterologous host cell which naturally lacks said genes.

The chemical modification of compounds (VI) can be used to produce the linked analogs discussed above. For example, the above-described pH-sensitive or redox potential-sensitive linkers can be attached to (VI) through an ester linkage to one of the hydroxyl groups of (VI), for example using a carbodiimide or active ester coupling.

In another aspect of the invention, novel cytotoxin-targeting molecule conjugates are provided. In one embodiment, the cytotoxin is maytansine linked through a C9-acetal moiety to an acid-sensitive linker that is attached to the targeting molecule. In a second embodiment, the cytotoxin is maytansine linked through a C9-thioacetal moiety to an acid-sensitive linker that is attached to the targeting molecule. In a third embodiment, the cytotoxin is maytansine linked through a C9-animal moiety to an acid-sensitive linker that is attached to the targeting molecule. In another embodiment, the cytotoxin is maytansine linked through the nitrogen atom of the C8-carbamate moiety to an acid-sensitive linker that is attached to the targeting molecule.

In another embodiment, the cytotoxin is selected from the group consisting of ansamitocins or ansamitocin analogs, rhizoxin or a rhizoxin analog, laulimalide or a laulimalide analog, and tedanolide or a tedanolide analog. In a further embodiment, the cytotoxin is a protein toxin, such as ricin or diphtheria toxin.

In another aspect of the invention, novel cytotoxin-targeting molecule conjugates are provided wherein multiple molecules of the cytotoxin are attached to each molecule of the targeting molecule through a dendrimer. In one embodiment, each molecule of cytotoxin is linked to a dendritic spacer via an acid-sensitive or redox-sensitive linker, and the targeting molecule is stably connected to the dendrimer. In a second embodiment, both the cytotoxin and the targeting molecule are transiently linked to the dendrimer through acid-sensitive or redox-sensitive linkers.

In the above-described embodiments, the targeting molecule can be any molecule that directs the cytotoxin to a specific cell, tissue, or organ. Preferred examples of targeting molecules include but are not limited to antibodies, growth factors, and polysaccharides. Preferred examples of antibodies include but are not limited to antibodies directed against HER2/neu, epidermal growth factor receptor (EGFR), ErbB2, platelet-derived growth factor (PDGF) receptor, vascular endothelial growth factor receptor 2 (VEGFR2 or KDR), and insulin-like growth factor receptor (IGFR). In other preferred embodiments, the antibody is directed against other clinically relevant tumor markers, including but not limited to polymorphic epithelial mucin (MUC-1), the ovarian cancer-associated antigen CA125, or against the CD33 myeloid-differentiation antigen. Preferred examples of growth factors include but are not limited to epidermal growth factor (EGF), insulin-like growth factor (ILGF), vascular endothelial growth factor (VEGF), and platelet-derived growth factor (PDGF). The targeting molecule may also be a polysaccharide having a specific interaction with the target cell or tissue. Preferred examples include but are not limited to polysaccharide ligands for selectin receptors.

The present invention provides compositions of matter that are formulations of one or more active drugs and a pharmaceutically acceptable carrier. In one embodiment, the formulation comprises a novel ansamitocin analog of the invention. In another embodiment, the formulation comprises a novel ansamitocin-targeting molecule conjugate of the invention. In both of these embodiments, the active compounds may be free form or where appropriate as pharmaceutically acceptable derivatives such as prodrugs, and salts and esters of the inventive compound. The composition may be in any suitable form such as solid, semisolid, or liquid form. See Pharmaceutical Dosage Forms and Drug Delivery Systems, 5^{th} edition, Lippicott Williams & Wilkins (1991) which is incorporated herein by reference.

In general, the pharmaceutical preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

Where applicable, the inventive compounds may be formulated as microcapsules and nanoparticles. General protocols are described for example, by Microcapsules and Nanoparticles in Medicine and Pharmacy by Max Donbrow, ed., CRC Press (1992) and by U.S. Pat. Nos. 5,510,118; 5,534,270; and 5,662,883 which are all incorporated herein by reference. By increasing the ratio of surface area to volume, these formulations allow for the oral delivery of compounds that would not otherwise be amenable to oral delivery.

The inventive compounds may also be formulated using other methods that have been previously used for low solubility drugs. For example, the compounds may form emulsions with vitamin E or a PEGylated derivative thereof as described by WO 98/30205 and 00/71163 that are incorporated herein by reference. Typically, the inventive compound is dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration. Another strategy involves encapsulating the inventive compounds in liposomes. Methods for forming liposomes as drug delivery vehicles are well known in the art. Suitable protocols include those described for other relatively insoluble drugs by U.S. Pat. Nos. 5,683,715; 5,415,869, and 5,424,073 and by PCT Publication WO 01/10412, each of which is incorporated herein by reference. Of the various lipids that may be used, particularly preferred lipids for making encapsulated liposomes include phosphatidylcholine and polyethyleneglycol-derivatized distearyl phosphatidylethanolamine.

Yet another method involves formulating the inventive compounds using polymers such as biopolymers or biocompatible (synthetic or naturally occurring) polymers. Biocompatible polymers can be categorized as biodegradable and non-biodegradable. Biodegradable polymers degrade in vivo as a function of chemical composition, method of manufacture, and implant structure. Illustrative examples of synthetic polymers include polyanhydrides, polyhydroxyacids such as polylactic acid, polyglycolic acids and copolymers thereof, polyesters polyamides polyorthoesters and some polyphosphazenes. Illustrative examples of naturally occurring polymers include proteins and polysaccharides such as collagen, hyaluronic acid, albumin, and gelatin.

The amount of active ingredient that may be combined with the carrier materials to produce a single dosage form will vary depending upon the subject treated and the particular mode of administration. For example, a formulation for intravenous use comprises an amount of the inventive compound ranging from about 1 mg/mL to about 25 mg/mL, preferably from about 5 mg/mL to 15 mg/mL, and more preferably about 10 mg/mL. Intravenous formulations are typically diluted between about 2 fold and about 30 fold with normal saline or 5% dextrose solution prior to use.

Methods to Treat Cancer

In one aspect, the present invention provides methods for treating cancer or other diseases or conditions of cellular hyperproliferation using pharmaceutically acceptable forms of the inventive compounds. In one embodiment, the compounds of the present invention are used to treat cancers of the head and neck, which include tumors of the head, neck, nasal cavity, paranasal sinuses, nasopharynx, oral cavity, oropharynx, larynx, hypopharynx, salivary glands, and paragangliomas. In another embodiment, the compounds of the present invention are used to treat cancers of the liver and biliary tree, particularly hepatocellular carcinoma. In another embodiment, the compounds of the present invention are used to treat intestinal cancers, particularly colorectal cancer. In another embodiment, the compounds of the present invention are used to treat ovarian cancer. In another embodiment, the compounds of the present invention are used to treat small cell and non-small cell lung cancer. In another embodiment, the compounds of the present invention are used to treat breast cancer. In another embodiment, the compounds of the present invention are used to treat sarcomas, including fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma. In another embodiment, the compounds of the present invention are used to treat neoplasms of the central nervous systems, particularly brain cancer. In another embodiment, the compounds of the present invention are used to treat lymphomas which include Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma

The method comprises administering a therapeutically effective amount of an inventive compound to a subject suffering from cancer. The method may be repeated as necessary either to contain (i.e. prevent further growth) or to eliminate the cancer. Clinically, practice of the method will result in a reduction in the size or number of the cancerous growth and/ or a reduction in associated symptoms (where applicable). Pathologically, practice of the method will produce at least one of the following: inhibition of cancer cell proliferation, reduction in the size of the cancer or tumor, prevention of further metastasis, and inhibition of tumor angiogenesis.

The compounds and compositions of the present invention can be used in combination therapies. In other words, the inventive compounds and compositions can be administered concurrently with, prior to, or subsequent to one or more other desired therapeutic or medical procedures. The particular combination of therapies and procedures in the combination regimen will take into account compatibility of the therapies and/or procedures and the desired therapeutic effect to be achieved.

In one embodiment, the compounds and compositions of the present invention are used in combination with another anti-cancer agent or procedure. Illustrative examples of other anti-cancer agents include but are not limited to: (i) alkylating drugs such as mechlorethamine, chlorambucil, Cyclophosphamide, Melphalan, Ifosfamide; (ii) antimetabolites such as methotrexate; (iii) microtubule stabilizing agents such as vinblastin, paclitaxel, docetaxel, epothilone, and discodermolide; (iv) angiogenesis inhibitors; and (v) cytotoxic antibiotics such as doxorubicon (adriamycin), bleomycin, and mitomycin. Illustrative examples of other anti-cancer procedures include: (i) surgery; (ii) radiotherapy; and (iii) photodynamic therapy.

In another embodiment, the compounds and compositions of the present invention are used in combination with an agent or procedure to mitigate potential side effects from the inventive compound or composition such as diarrhea, nausea and vomiting. Diarrhea may be treated with antidiarrheal agents such as opioids (e.g. codeine, diphenoxylate, difenoxin, and loeramide), bismuth subsalicylate, and octreotide. Nausea and vomiting may be treated with antiemetic agents such as dexamethasone, metoclopramide, diphenyhydramine, lorazepam, ondansetron, prochlorperazine, thiethylperazine, and dronabinol. For those compositions that includes polyethoxylated castor oil such as Cremophor®, pretreatment with corticosteroids such as dexamethasone and methylprednisolone and/or H₁ antagonists such as diphenylhydramine HCl and/or H₂ antagonists may be used to mitigate anaphylaxis.

What is claimed is:

1. A compound of the formula
T—L—C
wherein
T is a targeting molecule;
L is a pH-sensitive or redox potential-sensitive linker; and
C is a cytotoxin.

2. A compound of claim 1, wherein the cytotoxin C is maytansine, a maytansine analog, or an ansamitocin or ansamitocin analog.

3. A compound of claim 1, wherein linker L is a molecule of formula (A) 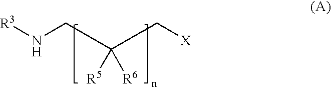
wherein
R³ is connected to targeting molecule T and is selected from the group consisting of 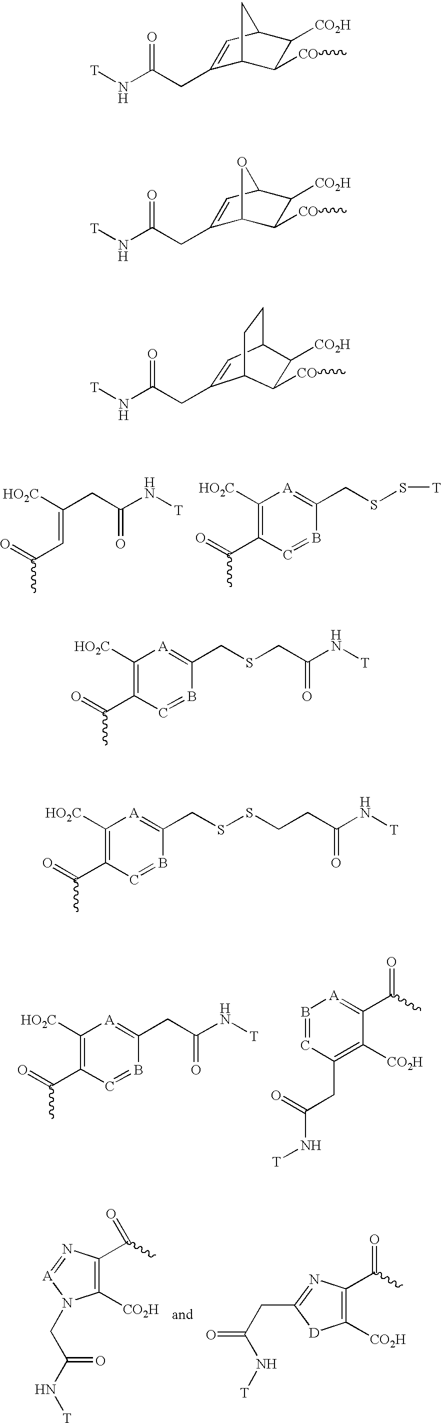
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁵ and R⁶ are independently H or methyl, or taken together form ═CH₂;
X is connected to the ansamitocin and is O, NH, S, O—(C═O)—O, S—(C═O)—O, O—(C═O)—S, S—(C═O)—S, O—(C═O)—NH, S—(C═O)—NH, or NH—(C═O)—NH; and
n=0, 1, 2, or 3.

4. A compound of claim 1, wherein linker L is a molecule of formula (B) 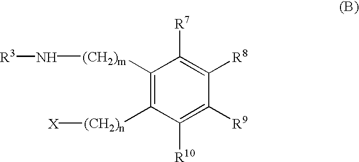
wherein
R³ is connected to targeting molecule T and is selected from the group consisting of 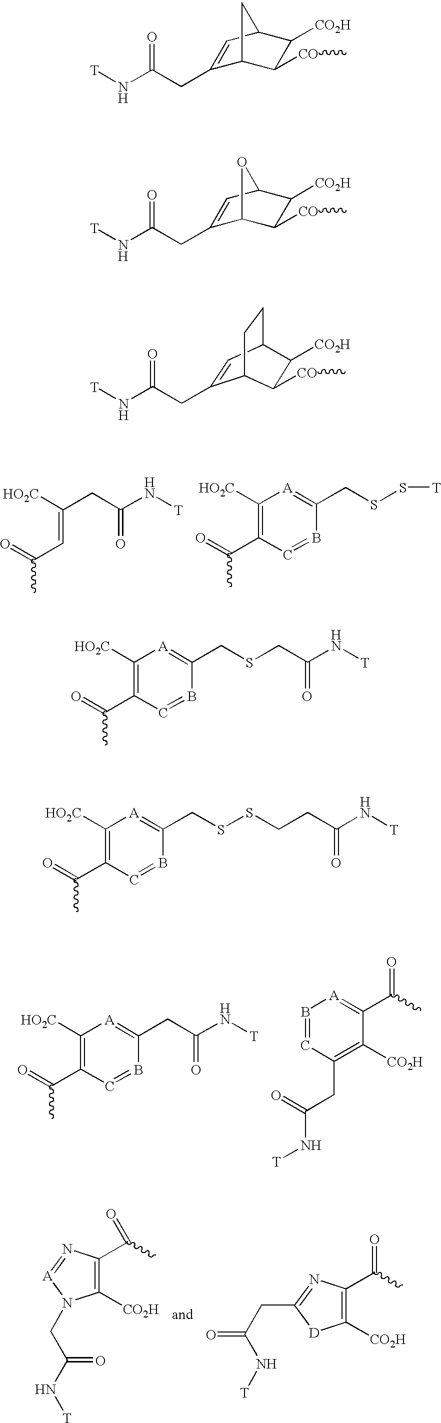
wherein A, B, and C are each independently N o r CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;
X is connected to the ansamitocin and is O, NH, or S; and
m and n are each independently 0, 1, 2, or 3.

5. A compound of claim 1, wherein linker L is a molecule of formula (C) 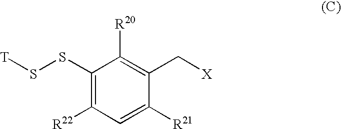
wherein
R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl; and
X is connected to the ansamitocin and is O, S, or NH.

6. A compound of claim 2 having formula (I) 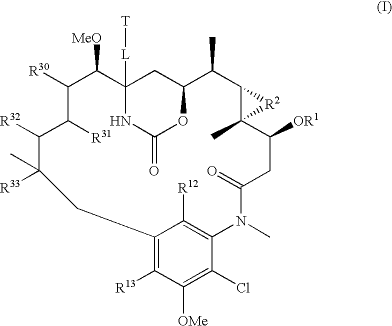
wherein
L is a pH-sensitive or redox potential-sensitive linker;
T is a targeting molecule;
R¹ is H, C(═O)R⁴, or C(═O)—CHMe—N(Me)—C(═O)—R⁴,
wherein R⁴ is C1-C6 straight or branched alkyl;
R is O or a bond;
R¹² and R¹³ are each independently H, OH, or NH2; and
R³⁰ is OH, R³¹ is H, and R³² and R³³ together form a bond, or R³² is OH, R³³ is H, and R³⁰ and R³¹ together form a bond.

7. A compound of claim 6, wherein linker L is a molecule of formula (A) 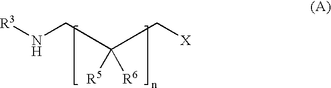
wherein
R³ is connected to targeting molecule T and is selected from the group consisting of 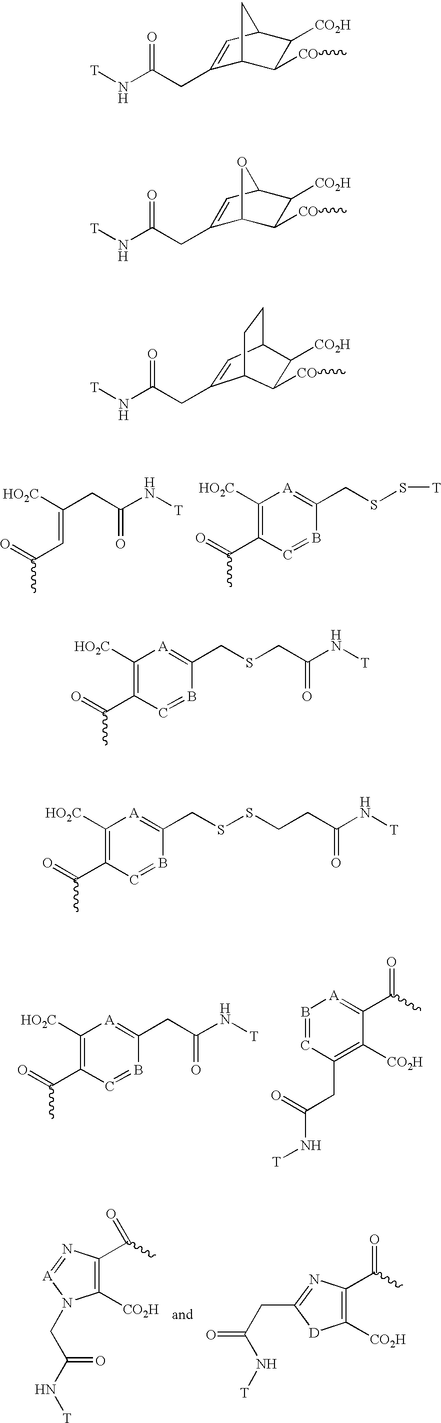
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁵ and R⁶ are independently H or methyl, or taken together form ═CH₂;
X is connected to the ansamitocin and is O, NH, S, O—(C═O)—O, S—(C═O)—O, O—(C═O)—S, S—(C═O)—S, O—(C═O)—NH, S—(C═O)—NH, or NH—(C═O)—NH; and
n=0, 1, 2, or 3.

8. A compound of claim 7, wherein targeting molecule T is an antibody, growth factor, or polysaccharide.

9. A compound of claim 6, wherein linker L is a molecule of formula (B) 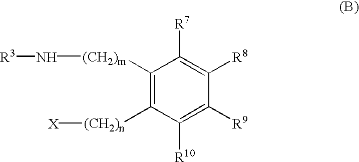
wherein
R³ is connected to targeting molecule T and is selected from the group consisting of 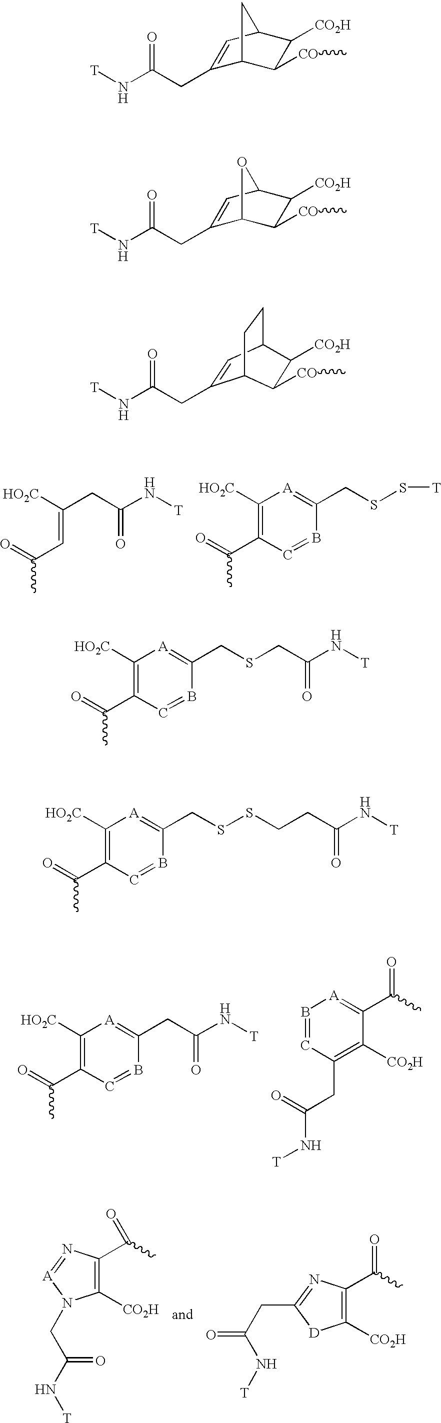
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;
X is connected to the ansamitocin and is O, NH, or S; and
m and n are each independently 0, 1, 2, or 3.

10. A compound of claim 9, wherein targeting molecule T is an antibody, growth factor, or polysaccharide.

11. A compound of claim 6, wherein linker L is a molecule of formula (C) 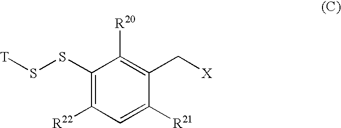
wherein
R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl; and
X is connected to the ansamitocin and is O, S, or NH.

12. A compound of claim 11, wherein targeting molecule T is an antibody, growth factor, or polysaccharide.

13. A compound of claim 2 having formula (II) 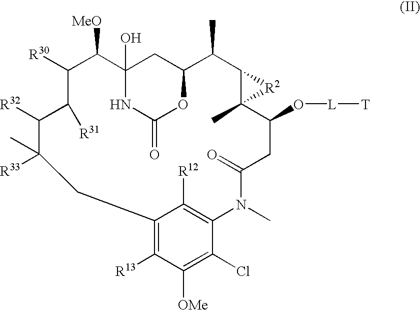
wherein
L is a pH-sensitive or redox potential-sensitive linker;
T is a targeting molecule;
R² is O or a bond;
R¹² and R¹³ are each independently H, OH, or NH2; and
R³⁰ is OH, R³¹ is H, and R³² and R³³ together form a bond, or R³² is OH, R³³ is H, and R³⁰ and R³¹ together form a bond.

14. A compound of claim 13 wherein linker L is a molecule of formula (D) 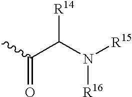
wherein
R¹⁴ is H or methyl;
R¹⁵ is H or methyl; and
R¹⁶ is taken from the group consisting of 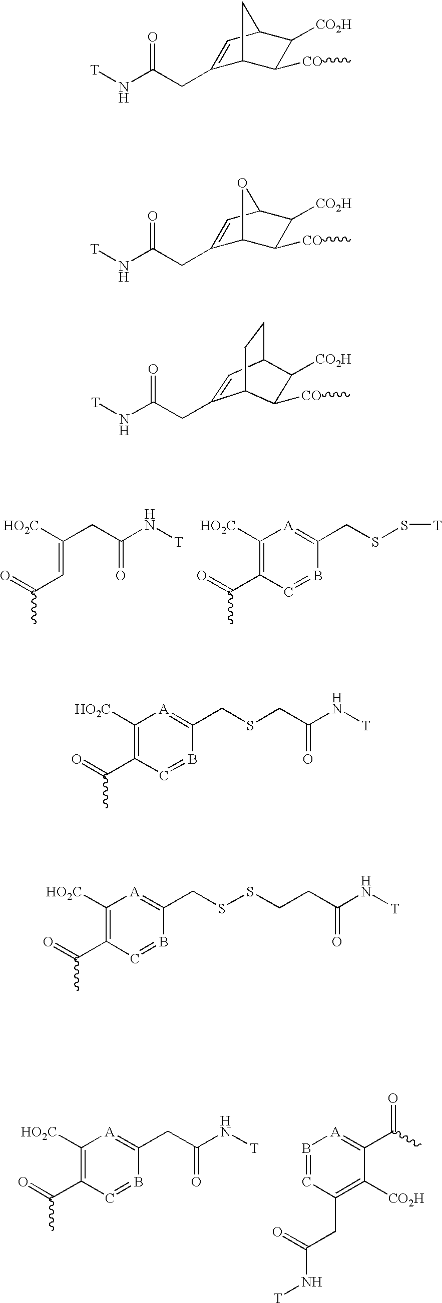
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl;
D is O, NH, or S; and
R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl.

15. A compound of claim 14 wherein the targeting molecule T is an antibody, growth factor, or polysaccharide. 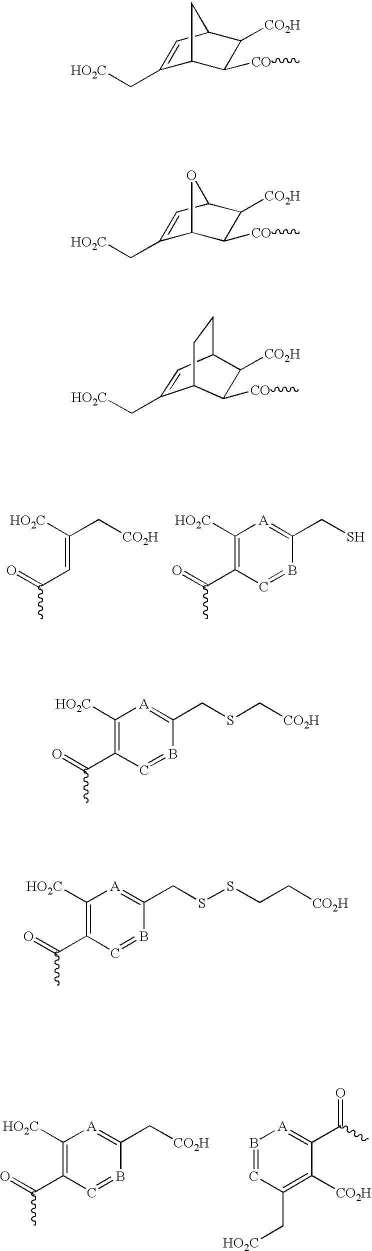
R² is O or a bond;
R¹² and R¹³ are each independently H, OH, or NH2;
R²⁰, R²¹, and R²² are each independently straight or branched C₁-C₄ alkyl, C₁-C₄ alkenyl, C₁-C₄ alkynyl, C₁-C₄ alkoxy, alktlthio, sulfonyl, alkylsulfonyl, halogen, amino, alkylamino, dialkylamino, nitro, cyano, formyl, carboxyl, carboxamido, or alkoxycarbonyl; and
R³⁰ is OH, R³¹ is H, and R³² and R³³ together form a bond, or R³² is OH, R³³ is H, and R³⁰ and R³¹ together form a bond.

16. A compound of the formula 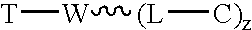
wherein
T is a targeting molecule;
W is a multivalent spacer of valency z;
L is a pH-sensitive or redox potential-sensitive linker;
C is a cytotoxin; and
z is from 1 to 128.

17. A compound of claim 16, wherein the cytotoxin C is maytansine, a maytansine analog, or an ansamitocin or ansamitocin analog.

18. A compound of claim 16, wherein linker L is a molecule of formula (A) 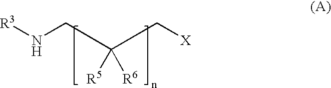
wherein
R³ is connected to multivalent spacer W and is selected from the group consisting of 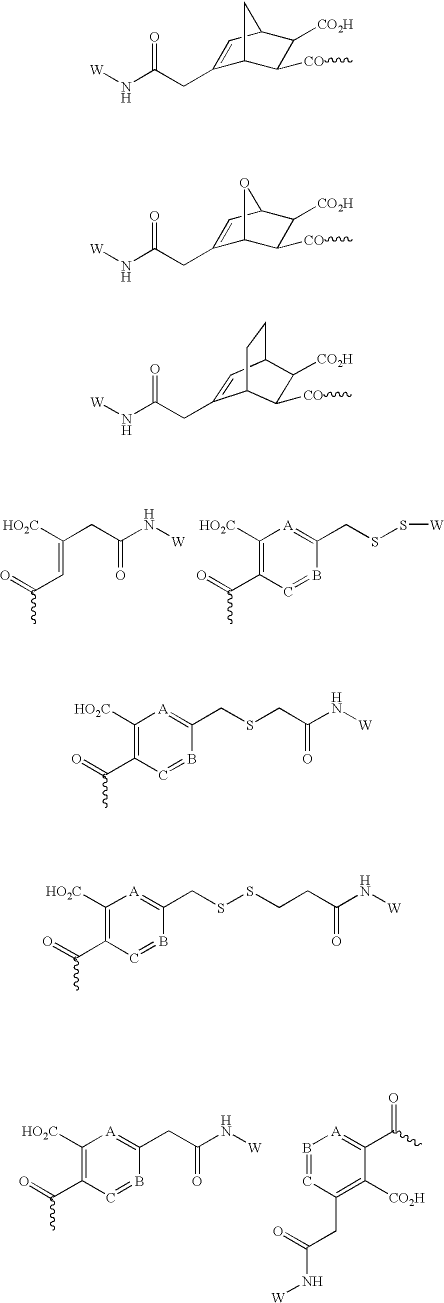
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁵ and R⁶ are independently H or methyl, or taken together form ═CH₂;
X is connected to the cytotoxin and is O, NH, S, O—(C═O)—O, S—(C═O)—O, O—(C═O)—S, S—(C═O)—S, O—(C═O)—NH, S—(C═O)—NH, or NH—(C═O)—NH; and
n=0, 1, 2, or 3.

19. A compound of claim 16, wherein linker L is a molecule of formula (B) 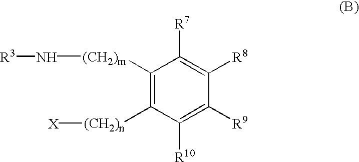
wherein
R³ is connected to multivalent spacer W and is selected from the group consisting of 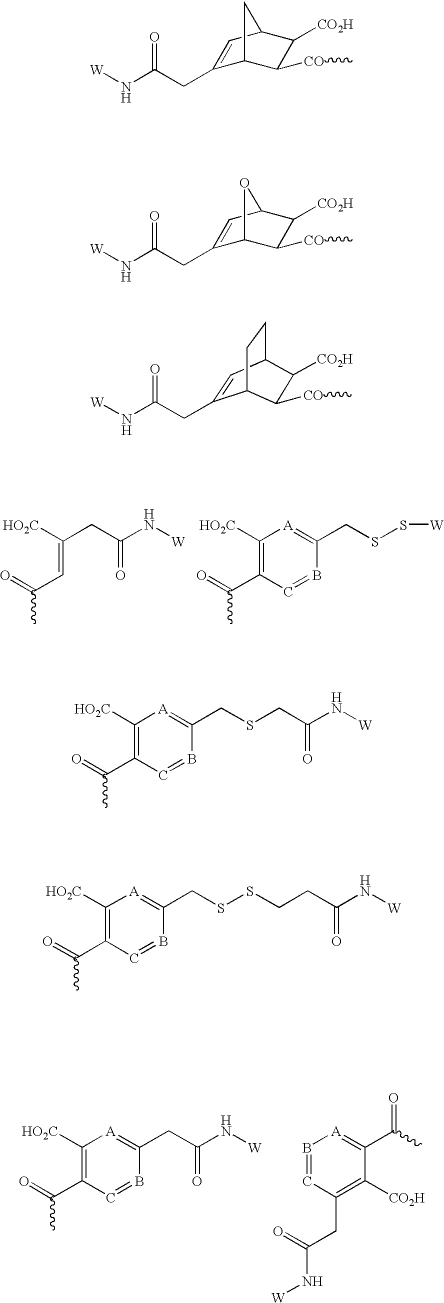
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;
X is connected to the cytotoxin and is O, NH, or S; and
m and n are each independently 0, 1, 2, or 3.

20. A compound of claim 16, wherein linker L is a molecule of formula (C) 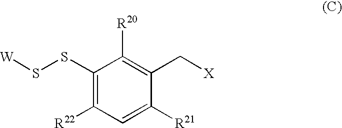
wherein
R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl; and
X is connected to the cytotoxin and is O, S, or NH.

21. A compound of claim 17 having the formula 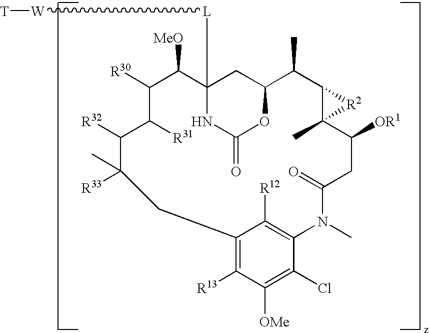
wherein
L is a pH-sensitive or redox potential-sensitive linker;
W is a multivalent spacer molecule of valency=z;
T is a targeting molecule;
R¹ is H, C(═O)R⁴, or C(═O)—CHMe—N(Me)—C(═O)—R⁴,
wherein R⁴ is C1-C6 straight or branched alkyl;
R² is O or a bond;
R¹² and R¹³ are each independently H, OH, or NH2;
R³⁰ is OH, R³¹ is H and R³² and R³³ together form a bond, or R³² is OH, R³³ is H, and R³⁰ and R³¹ together form a bond; and
z is from 1 to 128.

22. A compound of claim 21 wherein linker L is a molecule of formula (A) 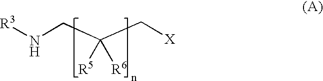
wherein
R³ is connected to one valency of multivalent spacer W and is selected from the group consisting of 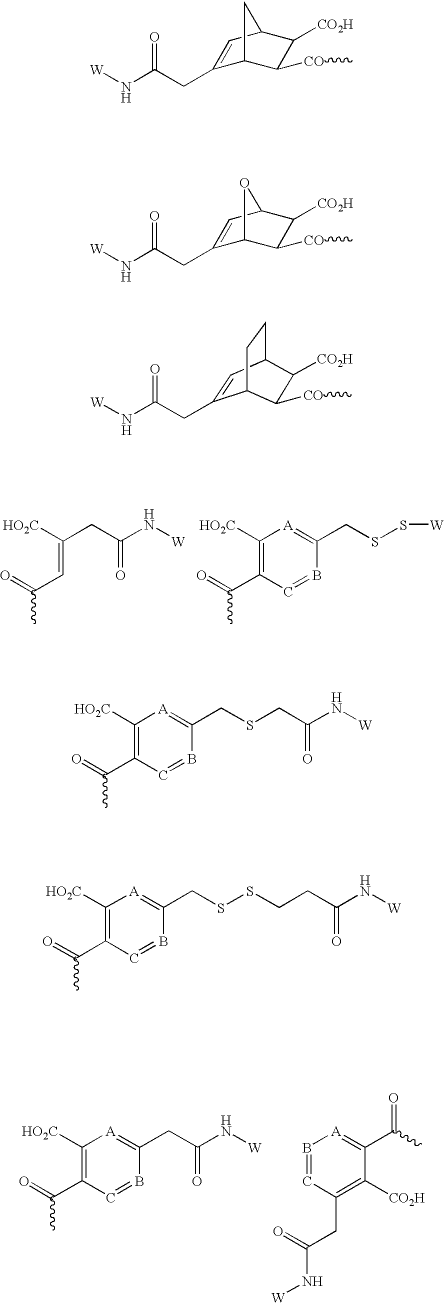
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁵ and R⁶ are independently H or methyl, or taken together form ═CH₂;
X is connected to the ansamitocin and is O, NH, S, O—(C═O)—O, S—(C═O)—O, O—(C═O)—S, S—(C═O)—S, O—(C═O)—NH, S—(C═O)—NH, or NH—(C═O)—NH; and
n=0, 1, 2, or 3.

23. A compound of claim 21 wherein linker L is a molecule of formula (B) 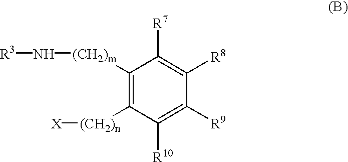
wherein
R³ is connected to one valency of multivalent spacer W and is selected from the group consisting of 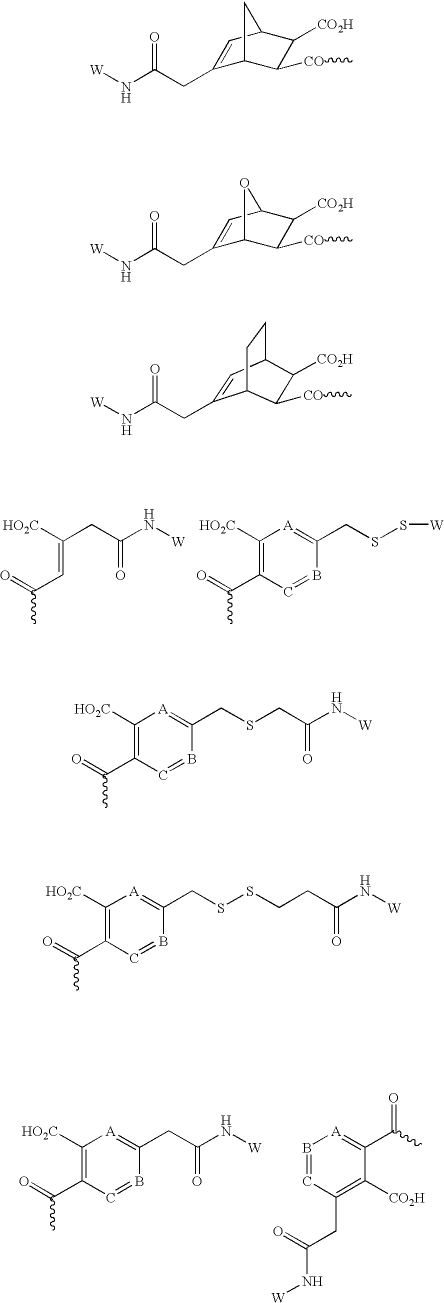
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl; and
D is O, NH, or S;
R⁷-R¹⁰ are each independently H, alkyl, alkenyl, alkynyl, aryl, halogen, hydroxy, carboxy, alkoxycarbonyl, alkylcarbonyl, formyl, nitro, amino, alkylamino, dialkylamino, alkylthio, alkoxy, or cyano;
X is connected to the ansamitocin and is O, NH, or S; and
m and n are each independently 0, 1, 2, or 3.

24. A compound of claim 22 or claim 23 wherein the multivalent spacer W is polylysine dendrimer or a StarBurst dendrimer.

25. A compound of claim 22 or claim 23 wherein the targeting molecule is an antibody, growth factor, or polysaccharide.

26. A compound of claim 17 having the formula 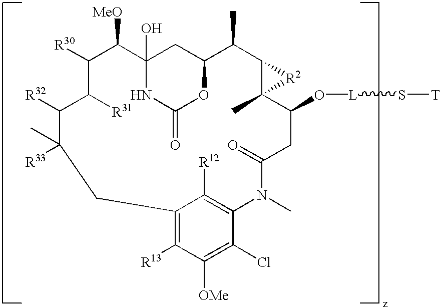
wherein
L is a pH-sensitive or redox potential-sensitive linker;
S is a multivalent spacer of valency=z;
T is a targeting molecule;
R₂ is O or a bond;
R¹² and R¹³ are each independently H, OH, or NH2;
R³⁰ is OH, and R³² and R³³ together form a bond, or R³² is OH, R³³ is H, and R³⁰ and R³¹ together form a bond; and
z is from 1 to 128.

27. A compound of claim 26 wherein the multivalent spacer W is polylysine dendrimer or a StarBurst dendrimer.

28. A compound of claim 26 wherein the linker L is a molecule of formula (D) 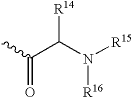
wherein
R¹⁴ is H or methyl;
R¹⁵ is H or methyl; and
R¹⁶ is connected to one valency of multivalent spacer W and is taken from the group consisting of 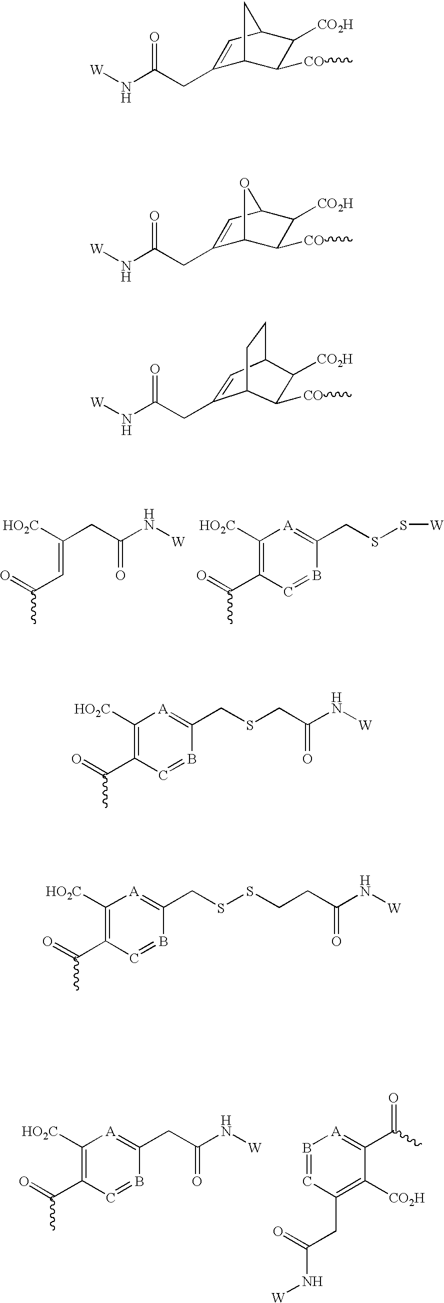
wherein A, B, and C are each independently N or CR¹⁹,
wherein R¹⁹ is selected from the group consisting of H, C₁-C₄ alkyl, alkoxy, hydroxy, amino, alkylamino, dialkylamino, halogen, nitro, cyano, carboxyl, alkoxycarbonyl, and formyl;
D is O, NH, or S; and
R²⁰, R²¹, and R²² are each independently straight or branched C₁-C₄ alkyl, C₁-C₄ alkenyl, C₁-C₄ alkynyl, C₁-C₄ alkoxy, alkylthio, sulfonyl, alkylsulfonyl, halogen, amino, alkylamino, dialkylamino, nitro, cyano, formyl, carboxyl, carboxamido, or alkoxycarbonyl.

29. A compound of claim 28 wherein the targeting molecule is an antibody, growth factor, or polysaccharide.

30. A method to treat a disease or condition of cellular hyperproliferation whereby a patient is provided with a compound of claims 1 or 16.

31. The method of claim 30 wherein the disease is cancer.

32. A compound of the formula 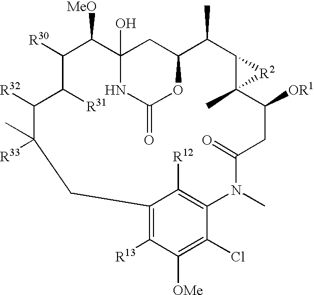
wherein
R¹ is H, C(═O)R⁴, or C(═O)—CHMe—N(Me)—C(═O)—R⁴,
wherein R⁴ is C1-C6 straight or branched alkyl;
R² is O or a bond;
R¹² and R¹³ are each independently H, OH, or NH₂; and
R³⁰ is OH, R³¹ is H, and R³² and R³³ together form a bond, or R³² is OH, R³³ is H, and R³⁰ and R³¹ together form a bond, or R³⁰ and R³¹ together form a bond and R³² and R³³ together form a bond;
with the proviso that when R³⁰ and R³¹ together form a bond and R³² and R³³ together form a bond that either R¹² or R¹³ are not H.

33. A compound of the formula 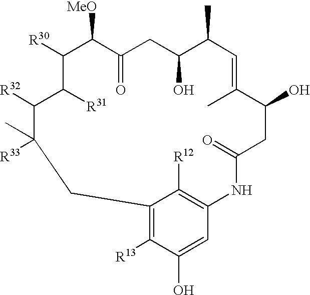
wherein
R¹² and R¹³ are each independently H, OH, or NH₂; and
R³⁰ is OH, R³¹ is H, and R³² and R³³ together form a bond, or R³² is OH, R³³ is H, and R³⁰ and R³¹ together form a bond, or R³⁰ and R³¹ together form a bond and R³² and R³³ together form a bond;
with the proviso that when R³⁰ and R³¹ together form a bond and R³² and R³³ together form a bond that either R¹² or R¹³ are not H.

34. A polyketide synthase that produces a compound of claim 33.

35. A plasmid containing a gene encoding a polyketide synthase of claim 34.

36. An organism containing a plasmid of claim 35.
